(19) United States
(12) Patent Application Publication
(10) Pub. No.: US 2025/0302415 A1
(43) Pub. Date: Oct. 2, 2025

(54) Individualized Whole-Lung Deposition Model

(71) Applicant: University of Iowa Research Foundation, Iowa City, IA (US)

(72) Inventors: Ching-Long Lin, Iowa City, IA (US); Xuan Zhang, Iowa City, IA (US)

(73) Assignee: University of Iowa Research Foundation, Iowa City, IA (US)

(21) Appl. No.: 18/864,277

(22) PCT Filed: May 9, 2023
(86) PCT No.: PCT/US2023/066791

§ 371 Date: Nov. 8, 2024

Related U.S. Application Data
(60) Provisional application No. 63/340,716, filed on May 11, 2022.

(51) Int. Cl. A61B 6/00; A61B 6/03; A61B 6/50; G06F 30/25; G06F 30/28; G06T 3/40; G06T 7/00; G06T 7/11; G06T 7/62; G16H 30/20

(52) U.S. Cl. CPC A61B 6/5217; A61B 6/032; A61B 6/50; A61B 6/5288; G06F 30/25; G06F 30/28; G06T 3/40; G06T 7/0016; G06T 7/11; G06T 7/62; G16H 30/20; G06T 2207/10081; G06T 2207/30061

(57) ABSTRACT

An innovative imaging-based subject-specific whole-lung deposition model is provided. Computed tomography (CT) lung volumetric images at total lung capacity (TLC) may be used to segment airways and lobes, and registration of CT images at TLC and functional residual capacity (FRC) provided metrics of regional air volume changes. A volume-filling technique may then be used to generate the entire conducting airways and acinar units. In each acinar unit, a respiratory airway model may be generated based on existing morphometric data. The flow distributions in conducting airways and to acinar units may be calculated by a one-dimensional (ID) computational fluid dynamics (CFD) model. With the simulated airflow field, deposition fractions may be calculated using deposition probability formulae adjusted with an enhancement factor to account for the effects of transient secondary flow and realistic airway geometry.

### RELATED APPLICATIONS

This application claims priority to U.S. Provisional Patent Application No. 63/340,716, filed May 11, 2022, entitled “Individualized Whole-Lung Deposition Model”, and hereby incorporated by reference in its entirety.

### FIELD OF THE INVENTION

The present invention relates to medical imagery. More particularly, but not exclusively, the present invention relates to methods and systems for modelling of a subject's respiratory tract to provide improved understanding, analysis, diagnosis, interventions, and/or treatment.

### BACKGROUND

The human respiratory tract is an important route for beneficial drug aerosol or harmful particulate matter to enter the body. To assess therapeutic response or disease risk, whole-lung deposition models were developed, but limited to compartment, symmetry or stochastic modeling. What is needed are new and innovative methods and system including a computed tomography (CT) imaging-based subject-specific whole-lung deposition model that may be used to assess the relationships between particle deposition patterns and airway structures in the whole lungs of individuals or subgroups characterized by distinct risk factors and/or lung disease stages.

### SUMMARY

Therefore, it is a primary object, feature, or advantage of the present invention to improve over the state of the art.

It is a further object, feature, or advantage to provide a provide a model suitable for predicting whole-lung particle deposition in human lungs.

It is a still further object, feature, or advantage to provide CT imaging-based subject-specific modeling.

Another object, feature, or advantage is to allow for assessment of genetic (genetically-determined airway variants, dysanapsis), behavioral (e-cig), and environmental (PM2.5, coronavirus laden droplets) risk factors of the human lungs.

Yet another object, feature, or advantage is to assess lung health associated with inhaled aerosol.

A further object, feature, or advantage is to provide methods and models which enhance understanding of the factors contributing to the risk and response of the lungs in order to improve lifestyle and work-environment interventions.

A still further object, feature, or advantage is to provide methods and models which enhance understanding of the factors contributing to the risk and response of the lungs in order to improve efficacy of inhalational drug delivery, such as e-cig users and young COVID survivors and inhaler design or user instructions for subgroups.

One or more of these and/or other objects, features, or advantages of the present invention will become apparent from the specification and claims that follow. No single embodiment need provide each and every object, feature, or advantage. Different embodiments may have different objects, features, or advantages. Therefore, the present invention is not to be limited to or by any objects, features, or advantages stated herein.

According to another aspect, an innovative imaging-based subject-specific whole-lung deposition model is provided. Computed tomography (CT) lung volumetric images at total lung capacity (TLC) may be used to segment airways and lobes, and registration of CT images at TLC and functional residual capacity (FRC) provided metrics of regional air volume changes. A volume-filling technique may then be used to generate the entire conducting airways and acinar units. In each acinar unit, a respiratory airway model may be generated based on existing morphometric data. The flow distributions in conducting airways and to acinar units may be calculated by a one-dimensional (1D) computational fluid dynamics (CFD) model. With the simulated airflow field, deposition fractions may be calculated using deposition probability formulae adjusted with an enhancement factor to account for the effects of transient secondary flow and realistic airway geometry.

According to another aspect, a method for providing a CT imaging-based subject-specific whole-lung modelling includes steps of acquiring one or more CT lung images and generating at least one residual functional capacity (FRC) image and at least one total lung capacity (TLC) image from the one or more CT lung images of a subject, processing at a computing system to segment airways and lobes from the at least one TLC image, registering at the computing system the at least one TLC image and the at least one FRC image to estimate regional air volume changes at image-voxel levels, generating at the computing system subject-specific conducting airways and acinar units using the at least one TLC image and associating each terminal bronchiole with one of the acinar units, associating each acinar unit with corresponding image voxels to calculate air volume change between two lung volumes for each acinar unit, and performing at the computing system volume adjustment to rescale dimensions of conducting airways and respiratory airways from the at least one TLC image to a desired lung volume. The method may further include performing air flow modeling at the computing system using the dimensions of the conducting airways and the respiratory airways rescaled to the desired lung volume. The air flow modeling may be performed using a 1D computational fluid dynamics simulation. The method may further include performing 1D particle deposition modeling. The one or more CT lung images may include a first CT lung image acquired at inspiration and a second CT lung image acquired at expiration. The computing system may include one or more processors. The step of acquiring the one or more CT lung images may include acquiring one more CT lung images from CT scans of the subject. The method may further include generating a visual output showing results of the whole-lung modeling including the conducting airways and the respiratory airways at the desired lung volume. The whole-lung modeling may be performed for more than one dimension.

According to another aspect, a system is provided. The system includes a computing device comprising at least one processor, a plurality of instructions for execution by the computing device wherein the instructions are configured to process CT lung images including at least one TLC image and at least on FRC image to segment airways and lobes from at least one TLC image, register the at least one TLC image and the at least one FRC image to estimate regional air volume changes at image-voxel levels, generate subject-specific conducting airways and acinar units using the at least one TLC image and associate each terminal bronchiole with one of the acinar units, associate each acinar unit with corresponding image voxels to calculate air volume change between two lung volumes for each acinar unit, and perform volume adjustment to rescale dimensions of conducting airways and respiratory airways from the at least one TLC image to a desired lung volume. The plurality of instructions may be stored on a non-transitory machine readable medium. The plurality of instructions may be further configured to perform air flow modeling using the dimensions of the conducting airways and the respiratory airways rescaled to the desired lung volume. The air flow modeling may be performed using a 1D computational fluid dynamics simulation. The plurality of instructions may be further configured to perform particle deposition modeling. The particle deposition modeling may be one-dimensional particle deposition modeling. The one or more CT lung images may include a first CT lung image acquired at inspiration and a second CT lung image acquired at expiration. The plurality of instructions may further provide for generating a visual output showing results of the whole-lung modeling including the conducting airways and the respiratory airways at the desired lung volume.

According to another aspect, a method for generating an imaging-based subject-specific whole-lung deposition model comprises steps of: obtaining computed tomography (CT) lung volumetric images at total lung capacity (TLC), segment airways and lobes om the CT lung volumetric images at TLC, registering CT images at TLC and CT lung volume images at functional residual capacity (FRC) to provide metrics of regional air volume changes, applying volume-filling to generate conducting airways and acinar units, calculating flow distributions in conducting airways and to acinar units using a one-dimensional (1D) computational fluid dynamics (CFD) model, and calculating deposition fractions using deposition probability formulae adjusted with an enhancement factor to account for effects of transient secondary flow and airway geometry to thereby provide the imaging-based subject-specific whole-lung deposition model. The steps may be performed by a computing system executing a plurality of instructions.

### BRIEF DESCRIPTION OF THE DRAWINGS

Illustrated embodiments of the disclosure are described in detail below with reference to the attached drawing figures, which are incorporated by reference herein.

FIG. 1 illustrates probability distributions of subject-specific terminal bronchioles by generation and by lobe: left upper lobe (LUL), left lower lobe (LLL), right upper lobe (RUL), right middle lobe (RML) and right lower lobe (RLL). Lobar probabilities peak at generation 16 except RLL at 18. The trachea is counted as generation 0. For each generation there is a group of bars which from left to right are LUL, LLL, RUL, RML, RLL.

FIG. 2 is a schematic of the first four generations of an acinar unit.

FIG. 3 is an illustration of flow passing airway segment A in three different categories. The first category such as shown in panel (a) is for flow carrying no particles; the second category such as shown at top in panel (b), particle-laden flow entering and exiting segment A with particle deposition; the third category, particle-laden flow entering but not exiting A with particle deposition, such as shown on top in panels (d)-(g). Panels (a)-(f), inspiratory flow; panels (g)-(i), expiratory flow.

FIG. 4A illustrates an example of a low shape factor=1.04.

FIG. 4B illustrates an example of a high shape factor=2.9.

FIG. 5 illustrates one example of structure of a support vector regression used to calculate enhancement factor.

FIG. 6 is a schematic of the workflow of one example of a subject-specific ID CFPD model.

FIGS. 7A and 7B provide a flow chart of particle deposition calculation. FIG. 7A is a flow chart of deposition calculation during inspiration. FIG. 7B is a flow chart of deposition at the end of expiration.

FIG. 8 illustrates waveforms of tidal breathing and deep inhalation maneuvers employed by Hofemeier et al. (21) and Koullapis et al. (26).

FIG. 9 illustrates a comparison of total deposition fractions at different tidal volumes between our 1D CFPD results and those of Yeh and Schum (15).

FIG. 10A and FIG. 10B illustrate a comparison of deposition fractions between our 1D CFPD results and those of Preludium by Koullapis et al. (26). FIG. 10A illustrates 420 ml tidal breathing, FIG. 10B illustrates 1680 ml deep inhalation.

FIG. 11 illustrates a comparison of acinar deposition between our 1D acinar model results and 3D CFPD acinar simulations by Hofemeier et al. (21).

FIGS. 12A and 12B illustrate a comparison of deposition fractions between our 1D CFPD model and 3D deep lung model (DLM) by Koullapis et al. (26). IN=Inspiration, EX=Expiration. FIG. 12A provides results for 420 ml tidal breathing and FIG. 12B provides results for 1250 ml deep breathing. For each of the selected particle diameters there are two stacks, the first stack being 1D acinar model results (from top to bottom, 1D CFPD EX conducting, 1D CFPD EX respiratory, 1D CFPD IN respiratory, 1D CFPD IN conducting) and the second stack being 3D CFPD acinar simulations (from top to bottom, 3D DLM EX conducting, 3D DLM EX respiratory, 3D DLM IN respiratory, and 3D DLM IN conducting).

FIGS. 13A, 13B, 13C, 13D, 13E, and 13F show distributions of enhancement factors by region. FIG. 13A illustrates central airways such as trachea (generation 0), left main bronchus and right main bronchus (generation 1) and lobar bronchi (generation 2). FIG. 13B illustrates RUL. FIG. 13C illustrates LUL. FIG. 13D illustrates RIL. FIG. 13E illustrates LLL and FIG. 13F illustrates RLL. For each particle diameter the group of distributions are from the lowest generation to the higher generation.

FIG. 14 shows one regression result for enhancement factor based on SVR model.

FIG. 15A and FIG. 15B illustrate contributions by different deposition mechanism to total deposition at various particle sizes. “Brownian motion” and “Sedimentation at end” correspond to P_{D}^{P} and P_{S}^{P}, respectively, in Eq. (11), referring to deposition probabilities of non-penetrating particles calculated by pause equations as described in (19). In both FIG. 15A and FIG. 15B, for a particle size of 0.01, there is a small percentage of turbulent diffusion (bottom), a large amount of laminar diffusion (middle), and a lesser amount of Brownian motion (top). In contract for a particle size of 10 there is a large percentage of sedimentation (bottom), a smaller percentage of sedimentation at end (medium), smaller percentage of impaction (near top), and then a very small percentage of other deposition mechanisms.

FIG. 16 is a comparison of total deposition fractions predicted by the current subject-specific 1D CFPD with and without enhancement factor, Yeh and Schum (15) symmetric model and Asgharian et al. MPPD stochastic model (20).

FIG. 17 is a comparison of deposition fractions of the current 1D CFPD model with and without enhancement factor in each phase (inspiration vs. expiration) and region (conducting vs. respiratory/acinar), IN=Inspiration, EX=Expiration. For each particle diameter there are two stacks shown. In the left stack for each particle diameter are deposition fractions without enhancement EX conducting, without enhancement EX respiratory, without enhancement IN respiratory, without enhancement IN conducting (bottom). In the right most stack for each particle diameter is with enhancement EX conducting, with enhancement EX respiratory, with enhancement IN respiratory, and with enhancement IN conducting (bottom).

FIG. 18 illustrates deposition fractions by generation at different particle size.

FIG. 19 is a comparison of lobar particle deposition distributions between CT/SPECT and 1D CFPD model.

FIG. 20 is a comparison of total deposition fractions in CT/SPECT subgroups of GOLD 0-1 and GOLD 2-3.

FIG. 21 is a comparison of the coefficients of variation (CV) of particle distribution between 1D CFPD and SPECT data in subgroups of GOLD 0-1 and GOLD 2-3.

FIG. 22 is a comparison of 1D CFPD-predicted deposition fractions between a nonsevere-mild COPD subject and a moderate-severe COPD subjects.

FIG. 23 is a flow chart showing a methodology.

FIG. 24 is a bock diagram of a computing system configured to process CT lung images.

FIG. 25 is another example of a methodology.

### DETAILED DESCRIPTION

### I. Introduction

The human airways are the pathways for inhaled noxious particulate matter, or pharmacological aerosol. The alterations in airway structure due to genetic abnormalities, poor lung growth in early life and lung diseases may lead to differential deposition patterns of inhaled aerosol that could affect disease risk and therapeutic response. Thus, it is critical to understand the relationships between particle deposition patterns and airway structures in the whole lungs of individuals or subgroups characterized by distinct risk factors and/or lung disease stages.

Some examples of airway-structure risk factors include airway-branch variation and dysanapsis. Airway variants are associated with an increase in chronic obstructive pulmonary disease (COPD) prevalence among both non-smokers and smokers (1). Dysanapsis is associated with COPD incidence and lung functional decline (2, 3). With large data acquired by multi-center studies, machine learning has been applied to identify disease subgroups (subpopulation or clusters) using computed tomography (CT) metrics (4-6). For example, four cross-sectional clusters have been identified from current smokers (7) and former smokers (8), respectively, and four longitudinal clusters in former smokers have been identified (9). Eight types of latent traits among lung tissue patterns have also been extracted from CT lung images (10). The cluster-guided three dimensional (3D) subject-specific computational fluid and particle dynamics (CFPD) strategy has been employed to assess preferential particle deposition patterns in cluster-representative archetypes of severe asthmatics (11-13). However, the high computational cost of 3D subject specific CFPD hinders its application to large cohorts. Thus, there is a need to develop efficient subject-specific 1D deposition models (14) that allow for assessment of lung structure-deposition relationships in individuals and subpopulations.

Several theoretical models were developed to study the particle deposition in human lungs. Yeh and Schum (15) developed a one-dimensional (1D) airway model based on a silicone rubber replica cast of human tracheobronchial airways from a 60 year old male Caucasian (16). Yeh-Schum model assumed that branches are symmetric and dichotomous in 5 lobes. The model can be described as a typical path for whole lung (typical path symmetric) or 5 typical paths for lobes (5-lobe symmetric). The former assumes symmetry for all bifurcations, whereas the latter takes into consideration of intra-subject variation only for main and lobar bronchi in the first few generations. The demarcation between conducting and respiratory airways is fixed at a specific generation for the entire lung for the typical path symmetric model or for each lobe for the 5-lobe symmetric model. The deposition probability is computed using analytical formulae in straight cylindrical tubes for three basic deposition mechanisms: diffusion, sedimentation, and impaction.

Hofmann et al. (17, 18) developed a Monte-Carlo stochastic deposition model that selects randomly the geometry of branches one at a time along the path of an inhaled particle based on the statistics of morphometric data (16) and then calculates deposition probabilities as in (15, 19). As a consequence, it avoids reconstruction of the entire airway tree. Asgharian et al. (20) developed multiple-path particle dosimetry (MPPD) models comprising ten 5-lobe, asymmetric, tracheobronchial tree models using the measurement data (16) together with the above stochastic model. These models were used to represent individual healthy adult male subjects for the study of inter-subject deposition variability. It is noteworthy that the aforementioned typical-path symmetric, 5-lobe symmetric, stochastic and MPPD models are all based on the same morphometric data reported by Rabbe et al. (16).

Semi-empirical models introduced by the International Commission on Radiological Protection (ICRP) model (20) calculated radiation doses to the respiratory tract of workers resulting from the intake of airborne radionuclides. In the ICRP model, airway structure is divided into multiple ‘filter’ model including extrathoracic (nasal and oral part), bronchial, bronchiolar and alveolar-interstitial part. Particle deposition is calculated in each filter by the breathing routes. The deposition mechanism is split into aerodynamic deposition and thermodynamic deposition. Instead of calculation based on a detailed airway tree model, the ICRP model divides subjects into male, female and children, providing a quick estimation on deposition in each region.

3D CFPD has been employed to study the particle deposition in acinar models. In a recent study, Hofemeier et al. (21, 22) created a detailed 3D sub-acinar structure generated by the algorithm of Koshiyama and Wada (23) that captures the statistics of human acinar morphometry (24). Koullapis et al. (25, 26) introduced a deep lung model to simulate particle deposition in both conducting and acinar regions. The airway geometry in this model comprised ten distal generations of Yeh-Schum 5-lobe symmetric conducting airways coupled to multiple sub-acinar models—a variant of Hofemeier's 10-generation sub-acinus model (21, 22).

From in vivo and in vitro studies, Lippmann (27) and Stahlhofen et al. (28) derived simple analytical expressions for the deposition efficiencies of the nasal passages, larynx, upper and lower ciliated thoracic airways and the non-ciliated portion of the lungs. De Backer et al. (29) compared the particle deposition result from single photon emission computed tomography (SPECT) with 3D computational fluid dynamics (CFD) (without particle simulation) in CT-based airway models. The study showed that the lobar SPECT tracer concentration is highly correlated with the lobar airflow fraction used in CFD.

This work presents a CT imaging-based subject-specific ID whole-lung deposition model. This model uses CT lung images to generate entire subject-specific conducting airways and acinar units using a volume filling algorithm (30, 31). CT images acquired at inspiration and expiration are registered (32) to estimate regional air volume changes (33). In each acinar unit, a 1D respiratory airway model based on Weibel's acinar morphometric data (34) is generated with the assumption of isotropic alveolar wall expansion/contraction regulated by the CFD-predicted flow rate for each terminal bronchiole. With a given lung volume (LV), the airway dimensions are adjusted from TLC. The flow distributions (ventilations) are calculated by an in-house ID CFD lung model (35, 36) to determine branch-specific flow fractions for each subject. Deposition in each segment is calculated using established analytical formulae (15, 19) adjusted by an enhancement factor to account for the effects of transient secondary flow and airway geometry in the first 8 generations. The model is validated against existing in silico 1D whole-lung deposition models, in silico 3D CFPD studies and in vivo CT/SPECT data.

### II. Methods

a. Human Subject Data and Image Processing

Two datasets were used for this study. The post-bronchodilator CT lung image data acquired from the Multi Ethnic Study of Atherosclerosis (MESA) study were used for model development. The CT and SPECT image data acquired at the University of Iowa were used for model validation. The study protocols were approved by respective Institutional Review Boards. The demographic information of these subjects is shown in Table 1. The CT/SPECT subjects were patients with chronic obstructive pulmonary disease (COPD) with the Global Initiative for Chronic Obstructive Lung Disease (GOLD) stages of 0-3. CT images provide anatomical/structural data, whereas SPECT images provide functional data. Each CT/SPECT subject had three static CT scans at TLC, FRC and residual volume (RV), and one dynamic ventilation SPECT scan per visit. Technetium-99m (99mTc) sulfur colloid was used as the radiopharmaceutical for ventilation SPECT imaging.

TABLE 1
Demographic data for CT/SPECT subjects and MESA subjects.

| | | SPECT |
|---|---|---|
| | MESA | N = 8 for each |
| | N = 7 | visit, 3 visits |
| Age, yrs | 45.42 (4.72) | 64.10 (11.6) |
| BMI, kg/m² | 26.87 (4.62) | 27.68 (3.60) |
| Sex (Male %) | 0 | 75 |
| Groups | Hypo-anapsis: 3 | GOLD0: 6 |
| | Hyper-anapsis: 4 | GOD1: 3 |
| | | GOLD2: 10 |
| | | GOLD3: 5 |

Because the size of sulfur colloid is below 1 μm (37), the aerosol is expected to be transported deep into the lung (38). The lobar deposition fractions can be estimated by the distributions of the tracer activity of 99mTc sulfur colloid via co-registration of SPECT images and CT images at FRC whose lung volume is close to that of tidal breathing during dynamic SPECT imaging.

### B. Airway Models

### Conducting Airways

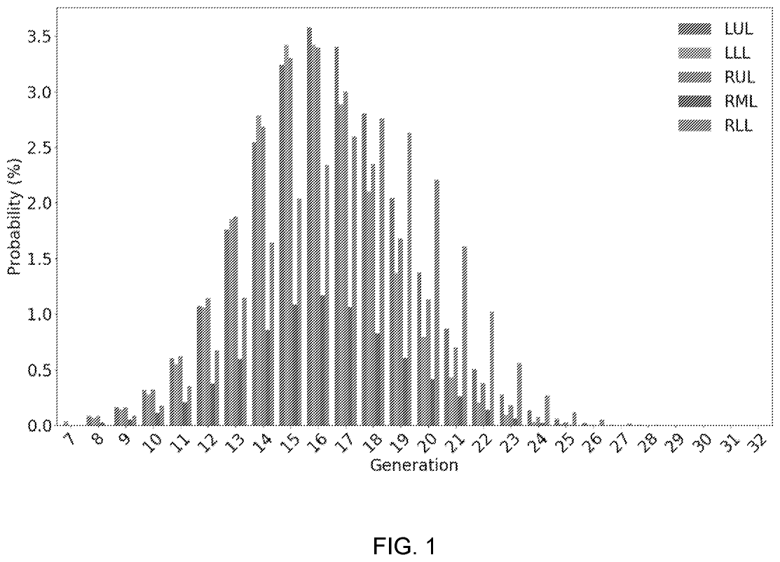

All CT scans were processed with a commercial software (VIDA Diagnostics) to segment the airways and lobes. A volume filling algorithm was employed to generate CT-based subject specific entire conducting human airway models (30, 31). The airway models are host-shape dependent because this algorithm uses five CT-segmented lobes as host-shaped boundaries and CT-resolved terminal airways as starting host-specific segments on an inspiration TLC scan that bifurcates repeatedly to supply around 30,000 acinar units distributed within five lobar cavities. The resulting CT-based airways models agreed with experimental morphometric data for normal subjects such as branching and length ratios, path lengths, numbers of branches, branching angles, and branching asymmetry (30, 31). For diseased lungs, we employed a stochastic airway narrowing model to determine the diameters of CT unresolved airways (35, 36). FIG. 1 shows the probabilities of generation numbers of terminal bronchioles in five lobes, which peak at around generation 16.c

Respiratory Airways with Isotropic Alveolar Wall Motion

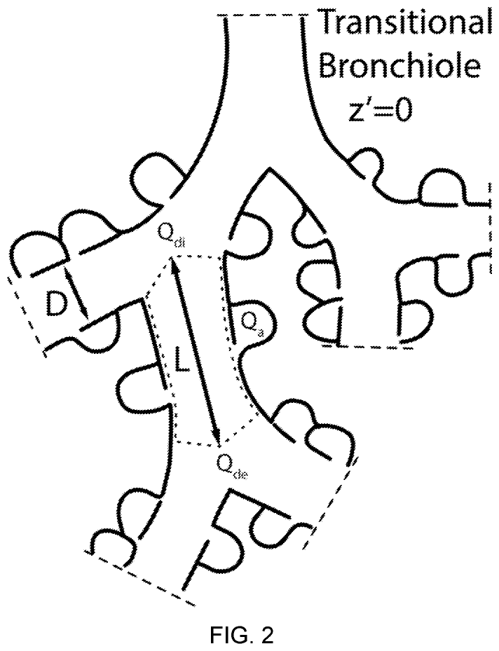

We employed an idealized typical-path model of acinar airways based on the measurements obtained on casts of human acinar airways by Weibel et al. (34) (Table 2 and FIG. 2). This dichotomously regularized model consists of 8 generations of acinar airways starting with a transitional bronchiole as the zeroth generation (z′=0), followed by three generations of respiratory bronchioles, four generations of alveolar ducts and one generation of terminal alveolar sacs, having an average volume of 187 cm³. The mean lengths and inner diameters of airway segments and the total alveolar surface at a given generation are used to derive the mean velocity inside each airway segment needed for the deposition formulae. The control volume inside a segment is outlined by the dashed lines. On inspiration, Q_{di} (or Q_{de}) is the flow rate at the inlet (or outlet) of an airway segment of generation z′. Qₐ is the flow rate into all alveoli at generation z′. The number of airway segments is N(z′)=2^{z′} Mass conservation at generation z′ yields Q_{de}·N=Q_{di}·N=Qₐ. With normalization by the flow rate at the transitional bronchiole Q_{di}(z′=0), we obtain:

$${q}_{de} \cdot N = {q}_{di} \cdot N - {q}_{a} \qquad ( 1 )$$

where qₖ≡Qₖ/Q_{di}(z′=0) with k=di, de and a; and hence q_{di}(z′=0)=1. With the assumption of isotropic alveolar wall expansion/contraction, qₐ(z′) is estimated as follows.

$${q}_{a} ( {z}^{′} ) = {S}_{alv} ( {z}^{′} ) {\overline{v}}_{alv} \qquad ( 2 )$$

where v̅ₐₗᵥ is the mean outward/inward normal velocity of the alveolar wall on expansion/contraction calculated by v̅ₐₗᵥ=1/Σ_{z′=0}⁸Sₐₗᵥ(z′) and Σ_{z′=0}⁸Sₐₗᵥ(z′)v̅ₐₗᵥ=q_{di}(z′=0)=1. After obtaining q_{de}(z′) via mass conservation, we then estimate the mean flow rate inside the airway segment, q̅_{d}=(q_{di}+q_{de})/2, and calculate q_{di}(z′+1)=q_{de}(z′)/2 for the next generation. The flow rate at the inlet of a transitional bronchiole Q_{di}(z′=0) (or the exit of a terminal bronchiole) is location and acinus specific, being derived by matching TLC and FRC images along with the imposed breathing wave form (see next section). The mean flow rate a, the mean airway length and the inner airway diameter are used to calculate deposition probabilities. It is noteworthy that the alveolar wall motion is assumed isotropic in each acinar unit, but is different among acini.

TABLE 2

| | | L | D | Sₐₗᵥ | | | | |
|---|---|---|---|---|---|---|---|---|
| z′ | N | (mm) | (mm) | (mm²) | qₐ | q_{di} | q_{de} | \grave{q}_{d} |
| 0 | 1 | 1.40 | 0.50 | 7 | 1.30E−03 | 1.00E+00 | 9.99E−01 | 9.99E−01 |
| 1 | 2 | 1.33 | 0.50 | 23 | 4.28E−03 | 4.99E−01 | 4.97E−01 | 4.98E−01 |
| 2 | 4 | 1.12 | 0.49 | 67 | 1.25E−02 | 2.49E−01 | 2.45E−01 | 2.47E−01 |
| 3 | 8 | 0.93 | 0.40 | 129 | 2.40E−02 | 1.23E−01 | 1.20E−01 | 1.21E−01 |
| 4 | 16 | 0.83 | 0.38 | 219 | 4.07E−02 | 5.99E−02 | 5.73E−02 | 5.86E−02 |
| 5 | 32 | 0.70 | 0.36 | 349 | 6.49E−02 | 2.87E−02 | 2.66E−02 | 2.77E−02 |
| 6 | 64 | 0.70 | 0.34 | 661 | 1.23E−01 | 1.33E−02 | 1.17E−02 | 1.25E−02 |
| 7 | 125 | 0.70 | 0.31 | 1204 | 2.24E−01 | 5.84E−03 | 3.95E−03 | 4.89E−03 |
| 8 | 256 | 0.70 | 0.29 | 2720 | 5.06E−01 | 1.98E−03 | 2.22E−16 | 9.88E−04 |

Table 2. Regularized dichotomous model of acinar airways from the transitional bronchiole (generation z′=0) and terminal alveolar sacs (generation z′=8) (34) z′, generation number in acinar airways; N, number of branches; L, mean length of segments; D, mean inner diameter of segments; Salv, total alveolar surface per generation (the sum of Sₐₗᵥ over all z′ is ρSₐₗᵥ=5379 mm²); qₐ(z′), normalized flow rate into all alveolar sacs per generation calculated by Sₐₗᵥv̅ₐₗᵥ where v̅ₐₗᵥ=1/ΣSₐₗᵥ and Σ_{z′}, Sₐₗᵥv̅ₐₗᵥ=1; q_{di}(z′), normalized flow rate at the inlet of an airway segment calculated by [q_{di}(z′−1)−qₐ(z′−1)]/N(n′); q_{de}(z′), normalized flow rate at the exit of an airway segment calculated by 2·q_{di}(z′+1); the mean normalized flow rate inside an airway segment q̅_{d} is (q_{di}+q_{de})/2. qₐ, q_{di} and q_{de}. are normalized flow rates by the flow rate at the transitional bronchiole Q_{di}(z′=0).

### Rescaling of Airway Dimensions

The airway dimensions segmented from TLC images need to be rescaled to a lung volume (LV) close to normal breathing. The scaling factor below is calculated based on the assumption (19) that both airway diameters and lengths in the respiratory region of the lung are proportional to the cube root of LV, while those in the conducting region are proportional to the square root of LV. The scaling factor for conducting airway diameters from TLC to desired LV reads:

$$ζ = \sqrt{\frac{{V}_{D , LV}}{{V}_{D , TLC}}} \qquad ( 3 )$$

where V_{D,Y} is the volume of dead air space at lung volume Y (19). The scaling factor for respiratory airway diameters from TLC to LV reads:

$${ζ}_{1} = \sqrt[3]{\frac{{V}_{R , LV}}{{V}_{R , TLC}}} \qquad ( 4 )$$

where V_{R,Y} is the volume of respiratory airways at lung volume Y (V_{R,Y}=Y−V_{D,Y}).

The diameters of terminal bronchioles and the volumes of acinar units generated by a volume filling technique vary locally, while the dimensions of transitional bronchioles and acinar volumes in Weibel's acinar model are fixed. To adjust the dimensions of Weibel's model locally, the volume of each acinar unit is assumed to be proportional to the cube of the diameter of the associated terminal bronchiole. The following scaling factors are used to adjust terminal bronchiolar diameters and acinar volumes with respect to those of Weibel's model.

$${ζ}_{2} = \frac{{d}_{eterminal bronchiole}}{{\overline{d}}_{terminal bronchioles}} \qquad ( 5 )$$

$${ζ}_{3} = \sqrt[3]{\frac{{\overline{V}}_{FV}}{{V}_{Weibel}}} \qquad ( 6 )$$

where d_{eterminal bronchiole}, is the diameter of a terminal bronchiole obtained by a volume filling algorithm and d̅_{terminal bronchioles} is the average diameter of terminal bronchioles. V̅_{FV} is the average volume of acinar units generated by a volume filling algorithm and V_{Weibel} is the volume of Weibel's acinar model. The formulae for rescaling the diameters (d) and lengths (l) of conducting and respiratory airways are:

$${d}_{conducting aiway , LV} = ζ {d}_{conducting aiway , TLC} \qquad ( 7 )$$

$${d}_{acinar aiway , LV} = {ζ}_{1} {ζ}_{2} {ζ}_{3} {d}_{acinar aiway , Weibel} \qquad ( 8 )$$

$${l}_{acinar aiway , LV} = {ζ}_{1} {ζ}_{2} {ζ}_{3} {l}_{acinar aiway , Weibel} \qquad ( 9 )$$

### Calculation of Particle Deposition During Breathing

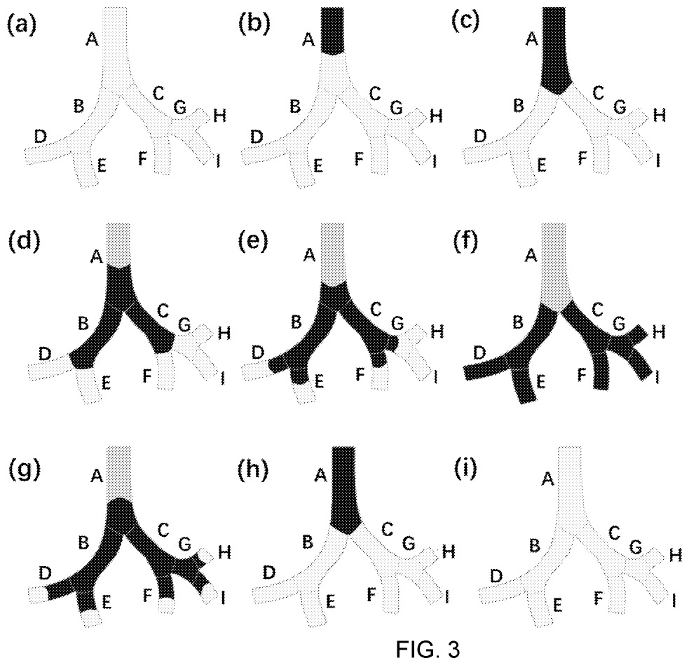

We employed an in-house 1D CFD model (33, 34) to calculate airflow in CT-based subject specific airways, and then calculated aerosol deposition probabilities for each airway segment due to turbulent/laminar/Brownian diffusion, sedimentation and inertial impaction (15, 19), denoted by P_{D}, P_{S} and P_{I}, respectively. On inspiration during the breathing cycle, the leading front of the particle-laden flow enters the trachea and penetrates into acinar units in a top-down order. On expiration, the flow is reversed, and residual particles exit the trachea in a bottom-up order. FIG. 3 illustrates the process of particle deposition during breathing. FIG. 3, panel (a) shows the initial state when the airways are filled with air. As inspiration continues, the particle laden flow starts to fill segment A in FIG. 3, panel (b). When the tip of the particle laden flow reaches the exist of segment A in FIG. 3, panel (c), the particle deposition in segment A is calculated as a combination of the probabilities of three mechanisms as follows:

$${P}_{inspiration} = {P}_{D} + {P}_{S} + {P}_{I} - {P}_{I} {P}_{D} - {P}_{I} {P}_{S} - {P}_{D} {P}_{S} + {P}_{I} {P}_{S} {P}_{D} \qquad ( 10 )$$

At the end of inspiration, a small portion of particles entering segment A cannot penetrate into its daughter branches B and C (FIG. 3, panels (d)-(f)). The deposition probability of these “non-penetrating” particles is calculated below using pause equations (15, 19) that depend on the duration of particles residing in segment A.

$${P}_{pause} = {{P}_{D}}^{P} + {{P}_{S}}^{P} - {{P}_{D}}^{P} {{P}_{S}}^{P .} . \qquad ( 11 )$$

where the superscript P denotes pause. During the expiration phase, the non-penetrating particles first exit segment A and those in the daughter branches B and C then pass through segment A as shown in FIGS. 3, panels (g)-(i). The deposition of particles in segment A is calculated as a combination of the probabilities of diffusion and sedimentation.

$${P}_{expiration} = {P}_{D} + {P}_{S} - {P}_{D} {P}_{S} \qquad ( 12 )$$

### Enhancement Factor

A turbulent laryngeal jet formed on inspiration at the downstream of glottal constriction (39, 40) and realistic (non-cylindrical) airway geometry can significantly increase particle deposition at bronchial bifurcations. Thus, the theoretically-derived analytical deposition probability formulae discussed above (15, 19) under-predict particle deposition in the central airways as being reported by (41). An enhancement factor defined as the ratio of actual total deposition probability to formulae-predicted total deposition probability is used to improve the accuracy of 1D deposition models. Previously, in vitro experimental data were used to calculate enhancement factors (17).

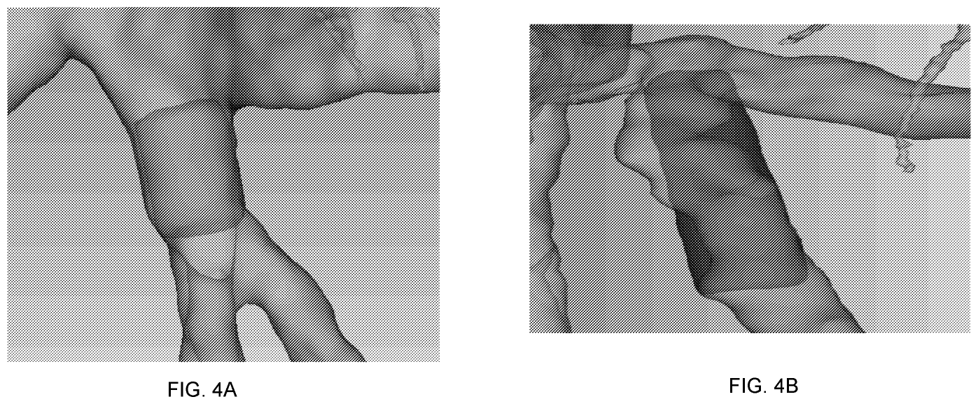

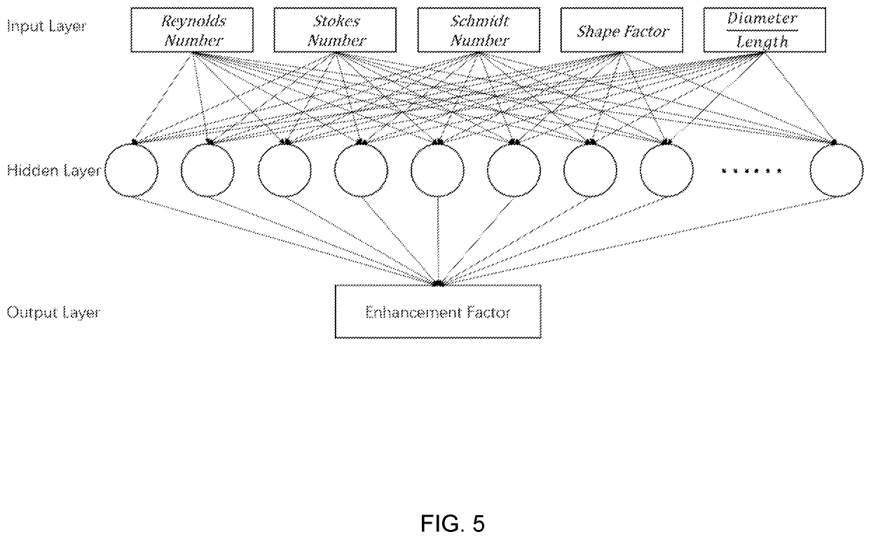

The enhancement factor applied only at inspiration phase as it had little effect at expiratory phase. Here, we used data generated by 3D CFPD simulations (12) to develop a regression model for prediction of enhancement factor for the first 8 generations of the airways starting from the trachea to improve deposition prediction accuracy. Specifically, we performed both 3D and 1D CFPD deposition simulations on the seven MESA subjects, and used 3D and 1D data to train a non-linear support vector regression (SVR) model for prediction of enhancement factor. By calculating the correlation of a number of dimensionless parameters with the enhancement factor, we found that Reynolds number, Stokes number, Schmidt number, shape factor and diameter-to-length ratio are good predictors for the enhancement of deposition in the first 8 generations of airways. The shape factor is to measure the extent of twisting, bending and narrowing of branches with respect to idealized cylindrical tubes. The shape factor of an airway segment is defined as the percentage of overlapping region between a CT-resolved airway and a straight cylindrical pipe defined by the start and end centerline points and the average diameter of the former. The range of shape factor is from 1 (same as a cylindrical pipe) to 4 (less than 25% of the image voxels are overlapped with a cylindrical pipe). FIGS. 4A and 4B show examples of low (FIG. 4A) and high (FIG. 4B) shape factors. In the SVR model, the variables are transformed into hyperplane in multidimensional space (feature space), and decision boundaries are found in the hidden layer to fit the data and predict the enhancement factor (FIG. 5).

### C. Modeling Process

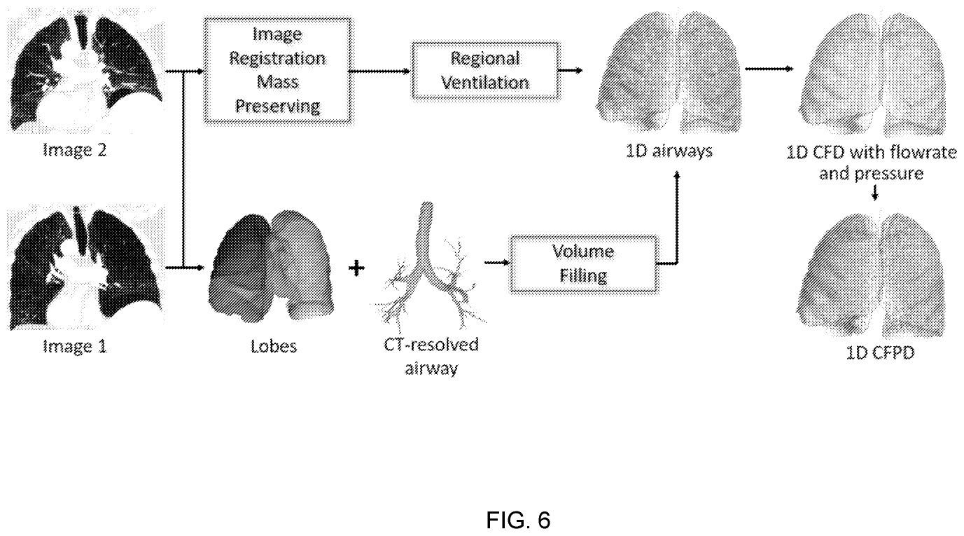

The 1D subject-specific CFPD modeling process consists of steps (a-f) below (see FIG. 6).
a. Image processing: Segment airways and lobes from TLC images, and register TLC and FRC images to derive air volume change between two lung volumes at image-voxel level.
b. Airway modeling: Generate entire subject-specific conducting airways and acinar units using TLC images and associate each terminal bronchiole with an acinar unit.
c. Regional ventilation: Associate each acinar unit with its corresponding image voxels to calculate air volume change between two lung volumes for each acinar unit.
d. Volume adjustment: Rescale dimensions of conducting airways and respiratory airways from TLC to desired LV.
e. Airflow modeling: Perform 1D CFD flow simulations.
f. Deposition modeling: Perform 1D deposition simulations.

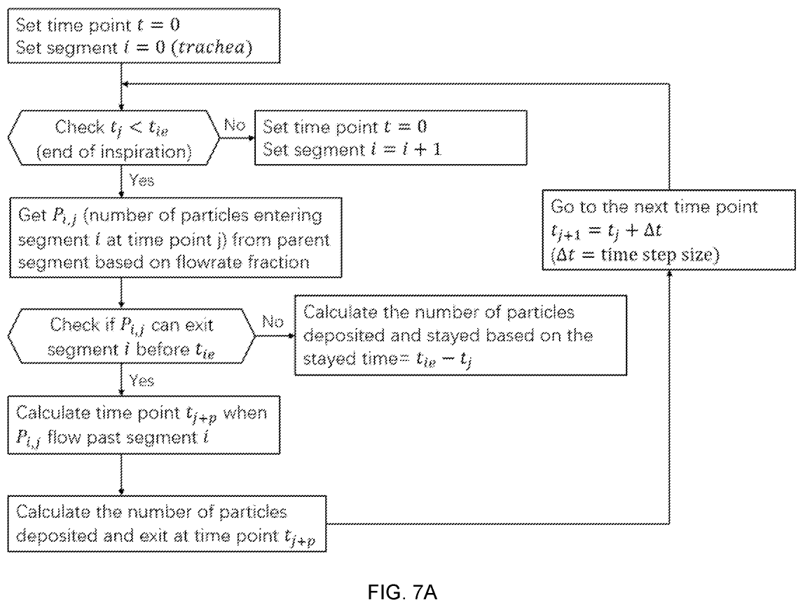

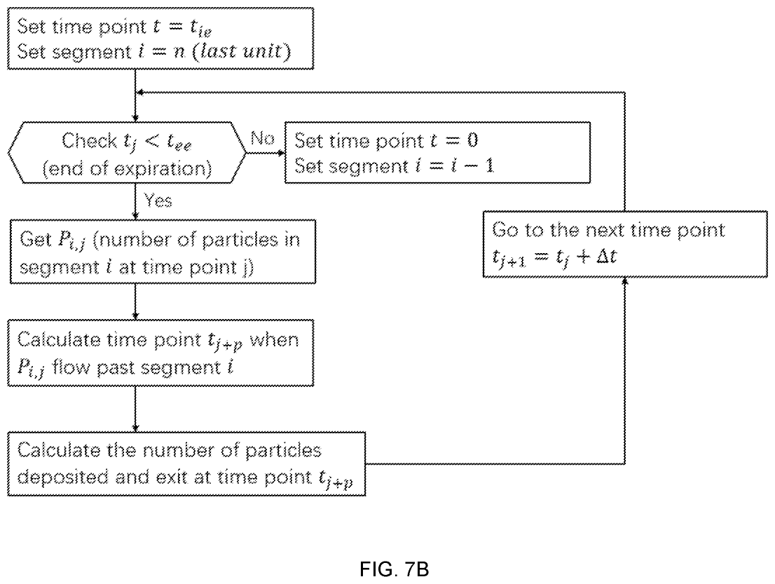

For step (a), we used the commercial software package VIDA Vision for segmentation and airway skeletonization and our in-house code (42, 43) for image registration. For step (b), we employed the volume filling technique (30, 31). For step (c), we followed the subject-specific modeling strategy (33). Step (d) was described in a previous section. For step (e), we used our in-house 1D CFD code (35, 36). Step (f) described before is summarized by a flow chart shown in FIG. 7.

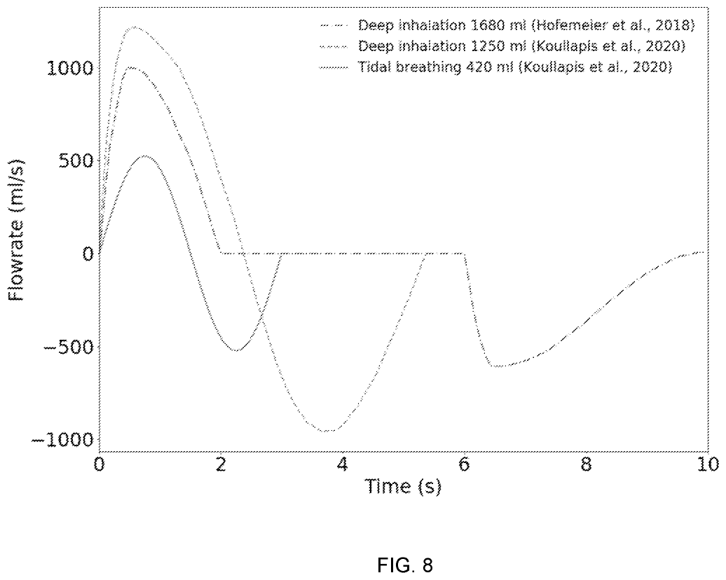

In this study, we imposed various waveforms to compare with previous studies (FIG. 8). The tidal breathing wave form is defined as:

$$Q ( t ) = {Q}_{peak} sin ( \frac{2 π}{T} t ) with {Q}_{peak} = \frac{π}{T} TV \qquad ( 13 )$$

where Qₚₑₐₖ is flowrate at peak inspiration, T is breathing period and TV is tidal volume. For subject-specific 1D CFPD, TV is estimated based on ideal body weight (IBW) (44).

$$IBW = C + 0.9 \cdot ( height [ cm ] - 152 ) , \qquad ( 14 )$$

where C=45.5 for female and 50.0 for male

$$TV = 7 kg / ml \cdot IBW \qquad ( 15 )$$

To compare with Hofemeier et al. (21) acinar model, deep inhalation waveform was employed. The deep inhalation waveform used 2.5 l as the breathing volume and a quick and deep waveform function to mimic rapid inhalation (Tᵢₙ=2 s). A 4 s breath hold (T_{BH}=4 s) fixed at constant volume followed the inspiration phase. The expiration phase had the similar function with the inspiration phase and the total breathing period was set to T=10 s. Koullapis et al. (26) used another deep inhalation waveform without breath hold. The waveform had a breathing volume of 1.68 l and a total breathing period of T=6 s.

### III. Results

### A. Validation Against 1D Symmetric Model Simulations

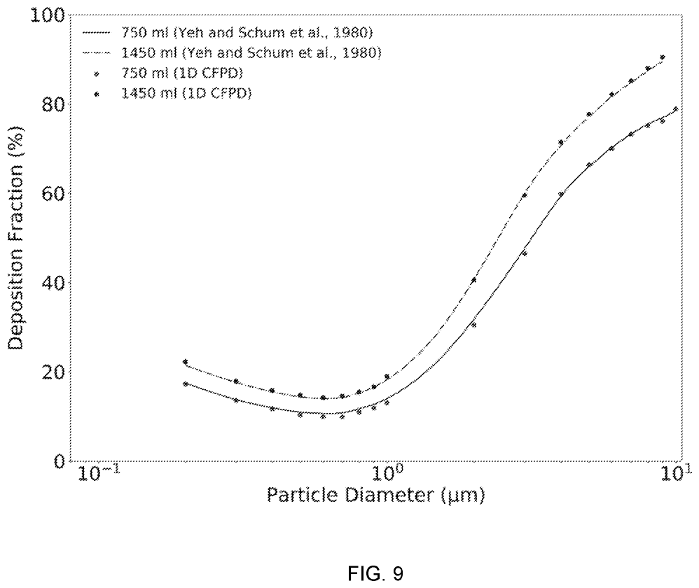

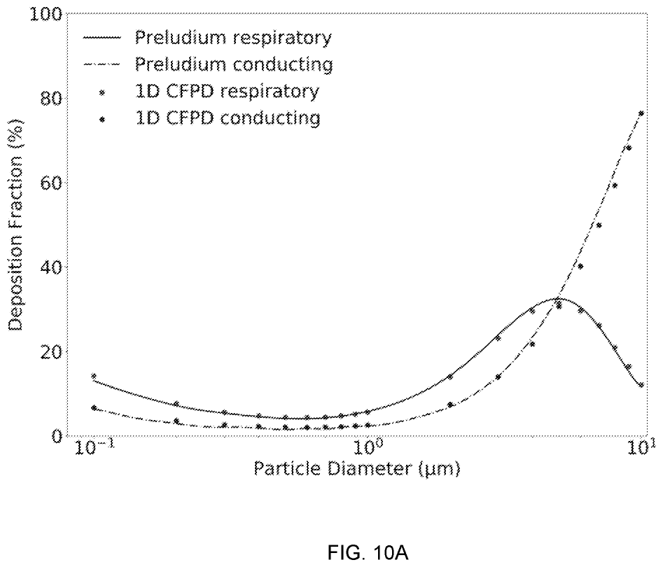

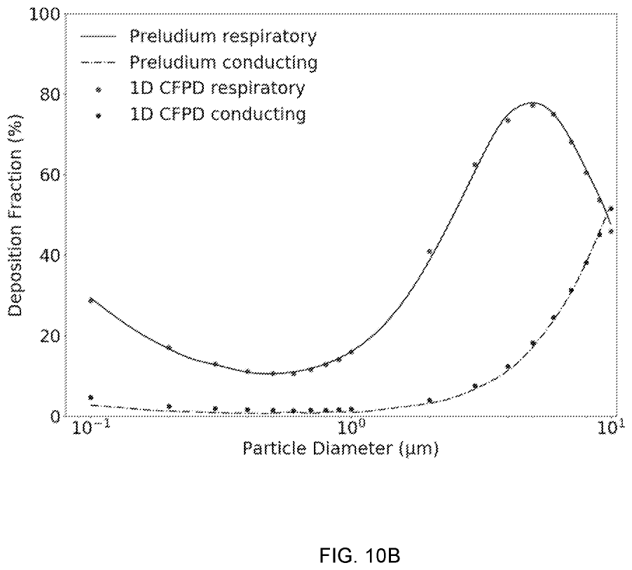

To verify and validate the time-sequence calculation of three deposition mechanisms in human lungs described above, we replicated the 1D deposition simulations of Yeh and Schum (15) using their typical-path symmetric airway model that did not include the mouth-throat region. Both 750 ml and 1450 ml tidal breathing waveforms were considered. The aerodynamics particle diameter varied from 0.1 to 10.0 μm. FIG. 9 shows good agreement between our results and those of Yeh and Schum (15). Moreover, we replicated the 1D deposition simulations of Koullapis et al. (26) calculated from a commercial software package Mimetikos Preludium that was based on Yeh-Schum's 5-lobe symmetric model. FIG. 10 shows good agreement between them for the waveforms of 420 ml tidal breathing and 1680 ml deep inhalation (see FIG. 8).

### B. Validation Against 3D CFPD Acinar Simulations

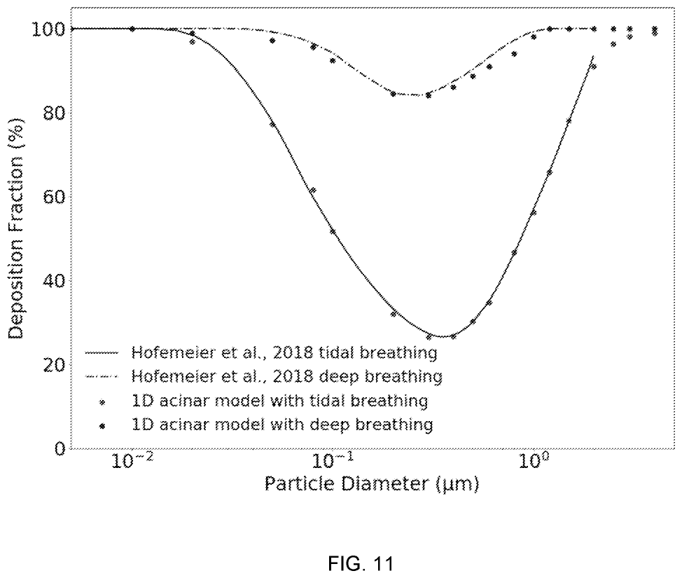

To validate our 1D acinar model, we calculated the deposition fractions in a single acinar unit with various waveforms and compared with those of 3D CFPD acinar simulations by Hofemeier et al. (21). It is noteworthy that our 1D acinar model was based on Weibel's morphometric data (34), whereas Hofemeier et al. adopted a detailed 3D sub-acinar structure generated by the algorithm of Koshiyama and Wada (23). Both 500 ml tidal breathing and 1250 ml deep breathing with a pause of 4 s (FIG. 8) were simulated. FIG. 11 shows good agreement between them for both waveforms in spite of discrepancies in acinar geometrical models.

### C. Validation Against 3D CFPD Deep Lung Model Simulations

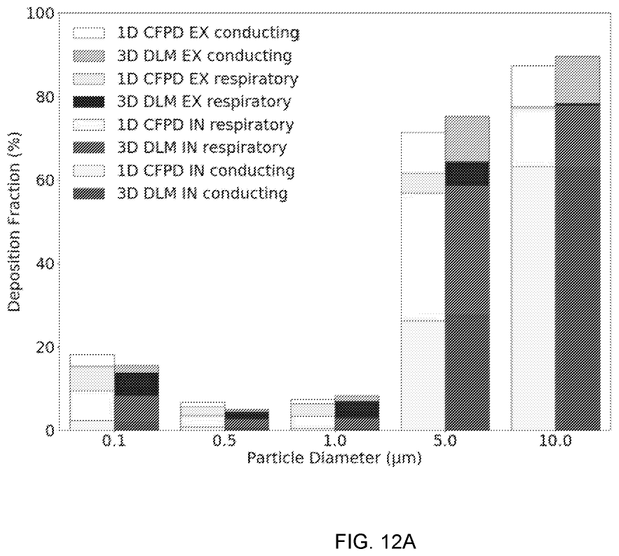

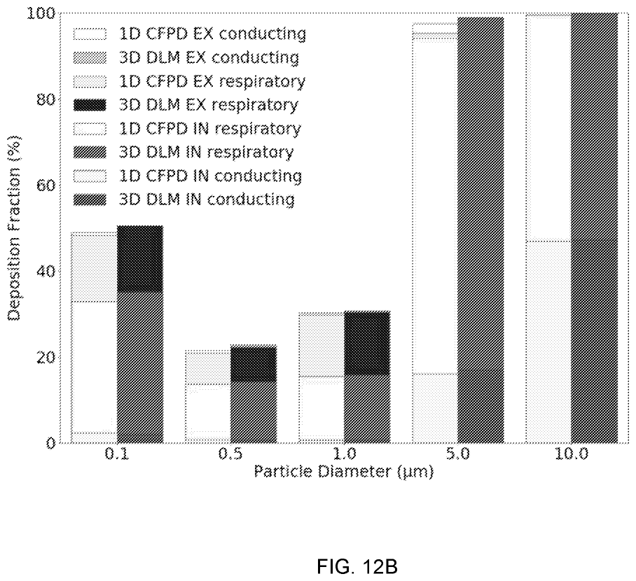

The 3D CFPD deep lung model developed by Koullapis et al. (26) comprised the last ten generations of Yeh and Schum's (15) symmetric conducting airways coupled to multiple subacinus units based on a variant of Hofemeier et al.'s acinar structure (21). To replicate their results for validation, we adopted the geometrical model of Yeh and Schum (15) for the last ten generations of conducting airways with each terminal bronchiole connected to a Weibel-based acinar model. An enhancement factor was not used because the 3D CFPD deep lung model only comprised distal airways. FIG. 12 shows good agreement between our 1D results and those of 3D CFPD deep lung model (26) over a wide range of particle sizes in conducting and respiratory regions on inspiration and expiration for both tidal breathing and deep inhalation (p>0.1).

### D. Enhancement Factor

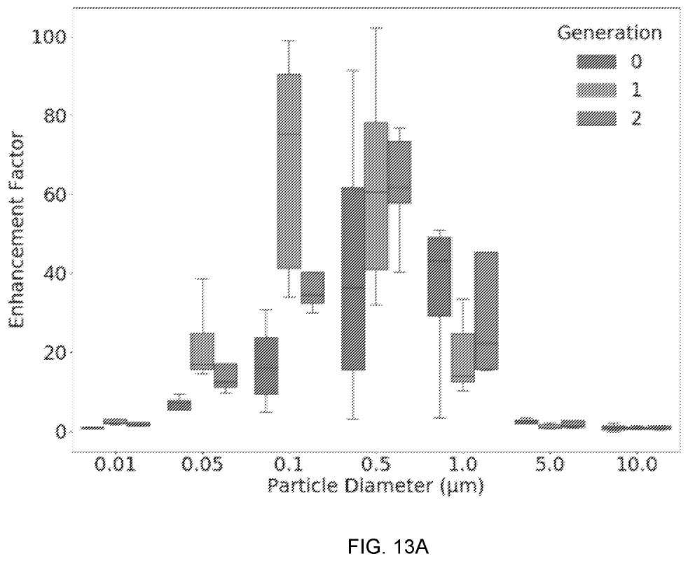

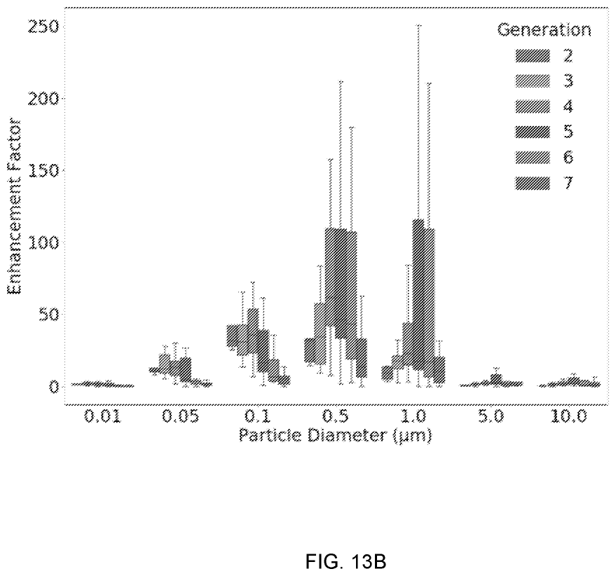

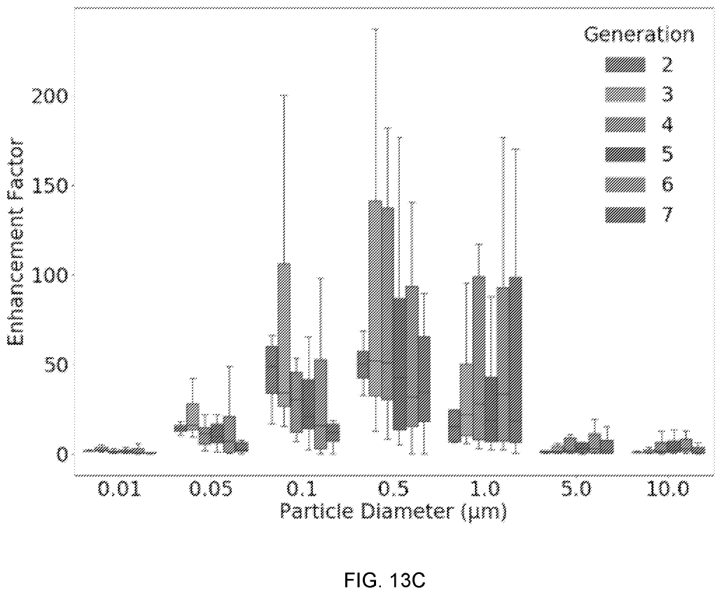

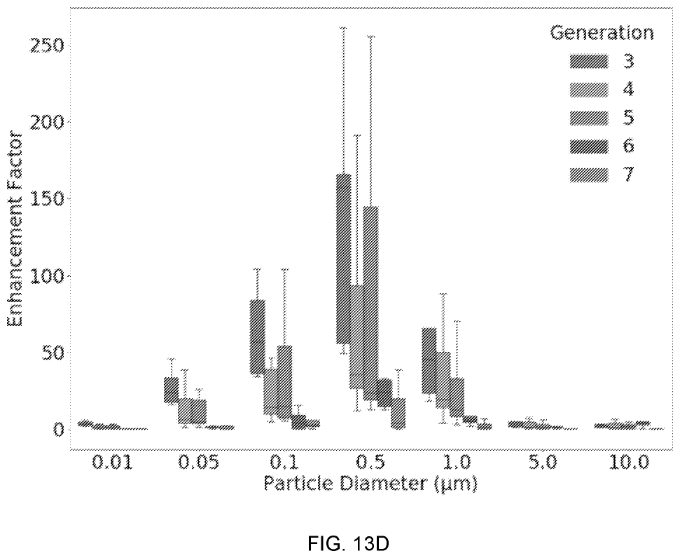

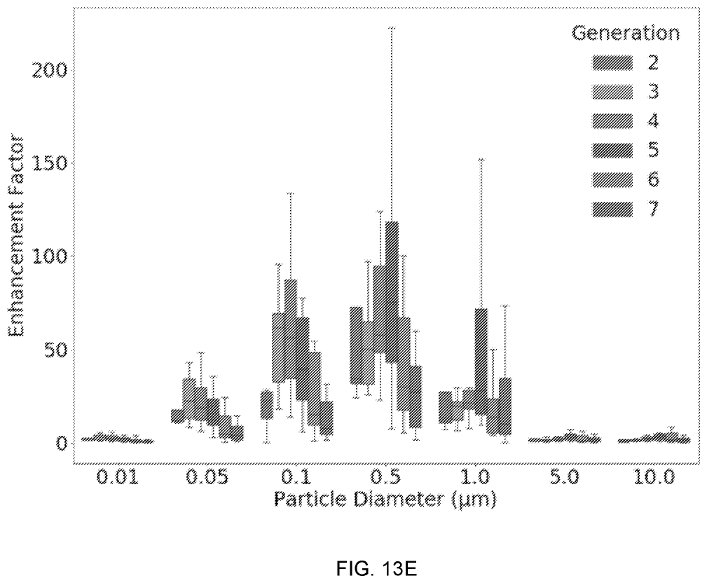

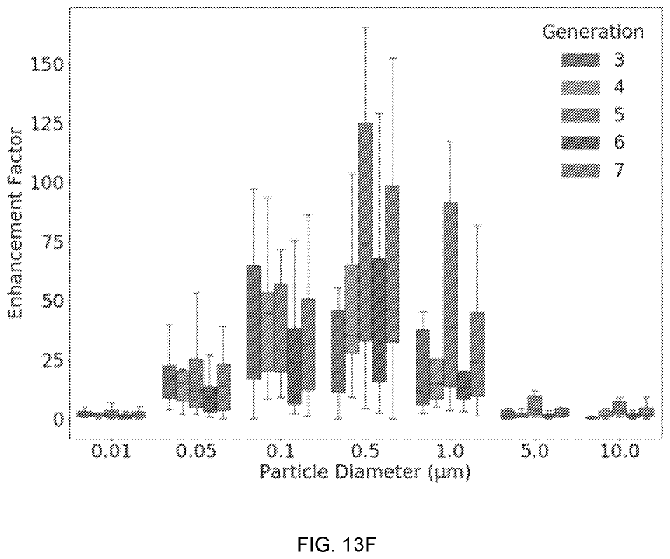

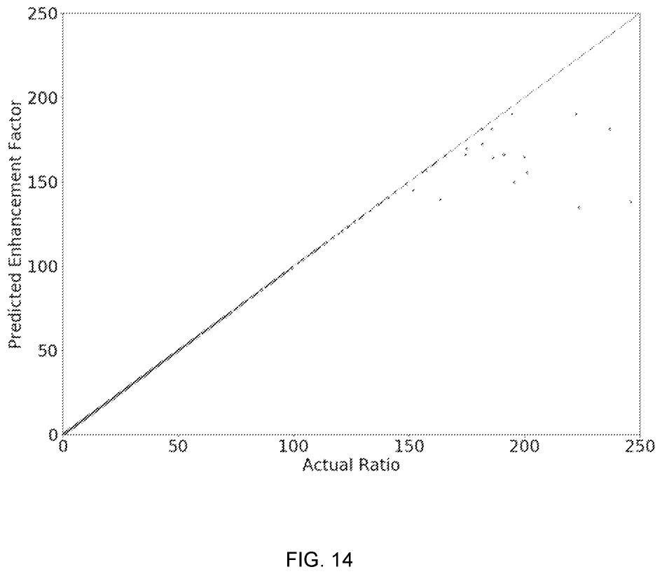

To calibrate the prediction of 1D model, we calculated the ratio of 3D CFPD deposition fractions over 1D deposition fractions for all branches of MESA subjects, called actual enhancement factor. FIG. 13 shows the distributions of actual enhancement factor in the first 8-generation proximal airways by particle diameter, region and airway generation. The results indicate that high enhancement factors center around particle diameter between 0.1 and 1 μm. FIG. 14 shows the comparison of SVR model-predicted enhancement factors with actual values. The agreement between them is good except for an enhancement factor greater than around 150 where airway segments are twisted or narrowed as measured by a shape factor, with r>0.7 (or r>0.9 by excluding the outliers having actual 3D/1D deposition ratio >150). Furthermore, we developed a simplified function below to predict the enhancement factor for inspiratory flow in airways at generation j based on particle size, shape factor and Reynolds number as:

$$Enhancement {factor}_{j} = α \cdot \frac{1}{{Re}_{peak}} {( shape factor )}^{\frac{1}{3}} \frac{l}{d} {e}^{\frac{{( {log}_{10} ( {D}_{ae} ) - β )}^{2}}{2 {γ}^{2}} + η} \qquad ( 16 )$$

Where α, β, γ and η are constants listed in Table 3. Reₚₑₐₖ is Reynolds number at peak inspiration with Reₚₑₐₖ≤4,000, and Dₐₑ is aerodynamic particle diameter. The empirical function yields r>0.6, compared to the SVR model of r>0.7.

TABLE 3
Table 3. Parameters for calculating enhancement factor.

| Generation | α | β | γ | η |
|---|---|---|---|---|
| 0 | 9,710 | −0.33 | 0.48 | 0.56 |
| 1 | 21,162 | −0.66 | 0.35 | 12.59 |
| 2 | 22,345 | −0.57 | 0.39 | 4.92 |
| 3 | 12,703 | −0.58 | 0.40 | 5.79 |
| 4 | 4,986 | −0.23 | 0.52 | 5.33 |
| 5 | 1,665 | −0.31 | 0.52 | 14.79 |
| 6 | 388 | −0.33 | 0.50 | 16.35 |
| 7 | 60 | −0.37 | 0.50 | 15.45 |

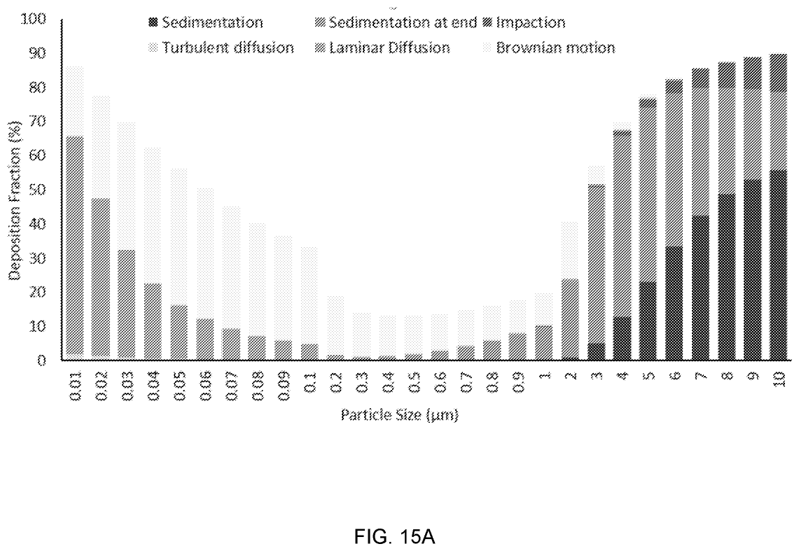

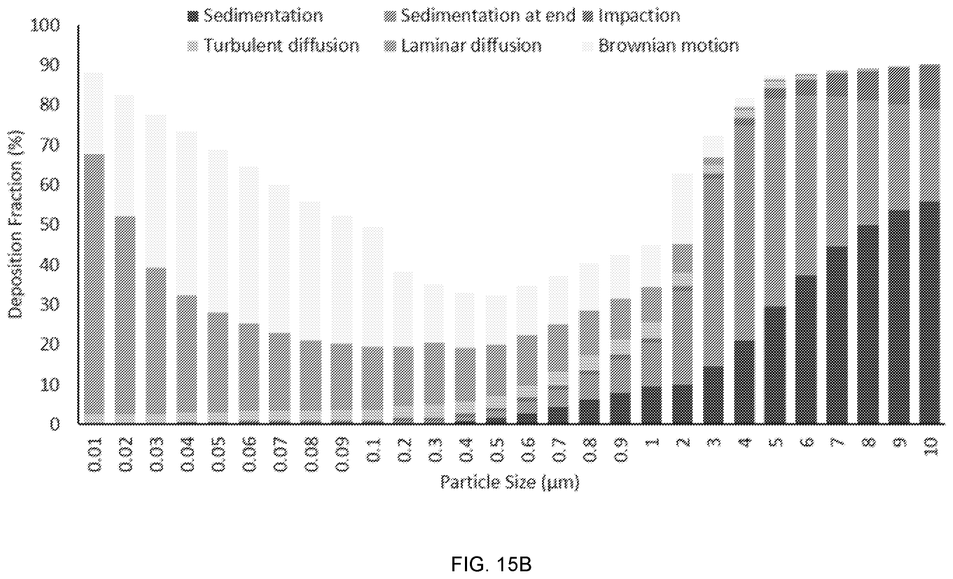

To inspect deposition mechanisms that were underestimated by probability formulae, FIG. 15 shows the breakdown of contributions by various mechanisms for cases without and with enhancement. For large particles of around 10.0 μm sedimentation is dominant, whereas for small particles approaching 0.01 μm diffusion plays a major role in distal airways. For particles between 0.1 and 1.0 μm, the enhancement factor has the most effect on laminar diffusion in proximal airways. Since the critical Reynolds number for transition from laminar flow to turbulent flow in a straight pipe was set to 2,300, the deposition probability formula for laminar diffusion in a straight pipe was used in most branches. This formula underestimated the deposition probably because it does not account for the effects of transient secondary flow in a branching network and real airway geometry.

### E. Subject-Specific Model Vs Symmetric/Stochastic Model

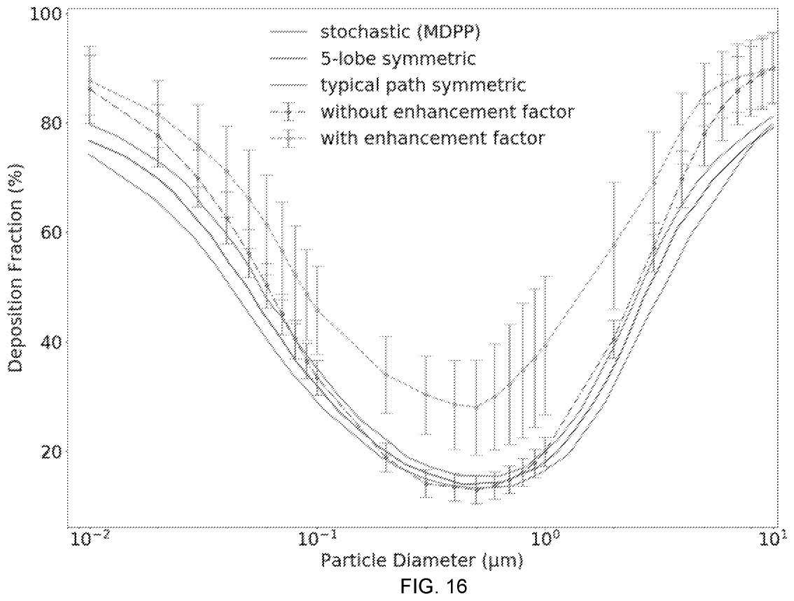

To compare the deposition features in subject-specific MESA subjects vs. symmetric/stochastic models, FIG. 16 shows the total deposition fractions computed by our 1D CFPD along with those of Yeh and Schum's symmetric model (15) and Asgharian's MPPD model (20) for particle diameter ranging from 0.01 to 10.0 μm. The 1D CFPD deposition fractions without enhancement resemble those of Yeh and Schum's and Asgharian's models (p>0.40) except for large (10.0-μm) and small (0.01-μm) particles with about 10% difference. The 1D CFPD results with enhancement show significant higher depositions than those without enhancement (p<0.05), particularly for particle diameter within the range of 0.1 and 1.0 μm.

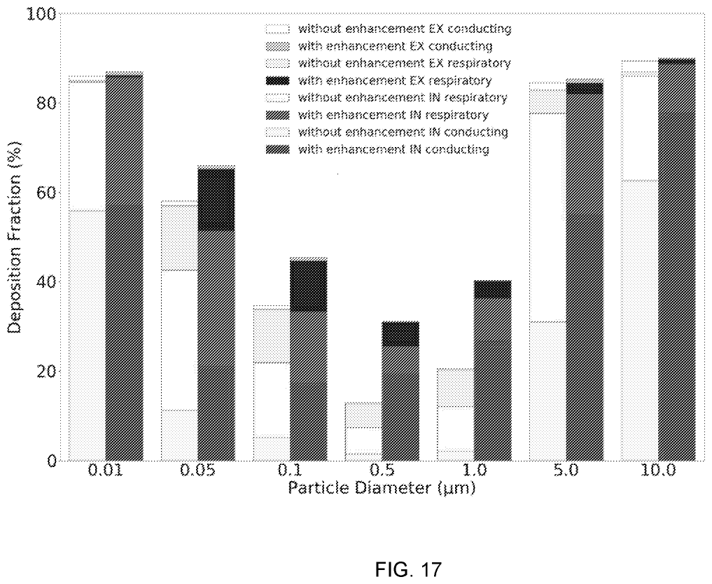

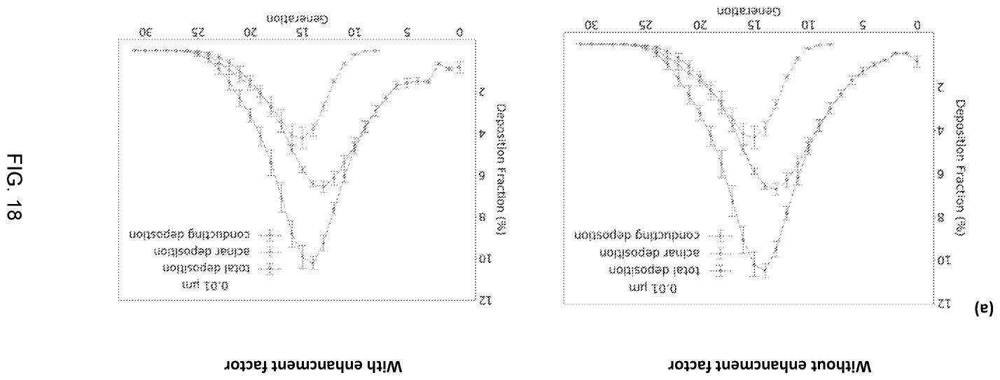

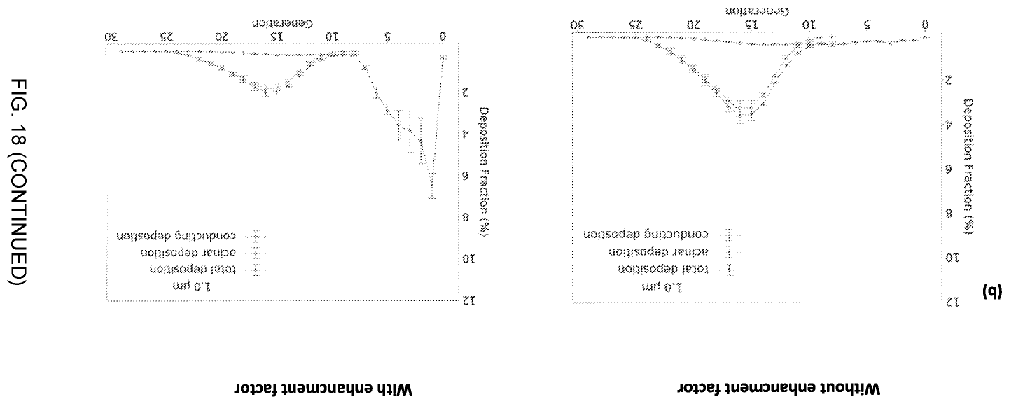

FIG. 17 shows a comparison of the 1D CFPD results with and without enhancement in conducting and respiratory airways on inspiration and expiration, respectively. The enhancement factor increases both the deposition in conducting airways on inspiration and the total deposition in the lungs. For 0.01 μm particles, the difference between total depositions with and without enhancement is insignificant because of small enhancement effect. For particle size ranging from 0.1 to 1.0 μm, the deposition on inspiration in conducting airways is enhanced due in large part to secondary flow and irregular geometry in the CT-based proximal airways. With increasing particle size to 10.0 μm, the total deposition fraction increases due to sedimentation and impaction, and most particles escaping conducting airways are deposited in respiratory airways on inspiration.

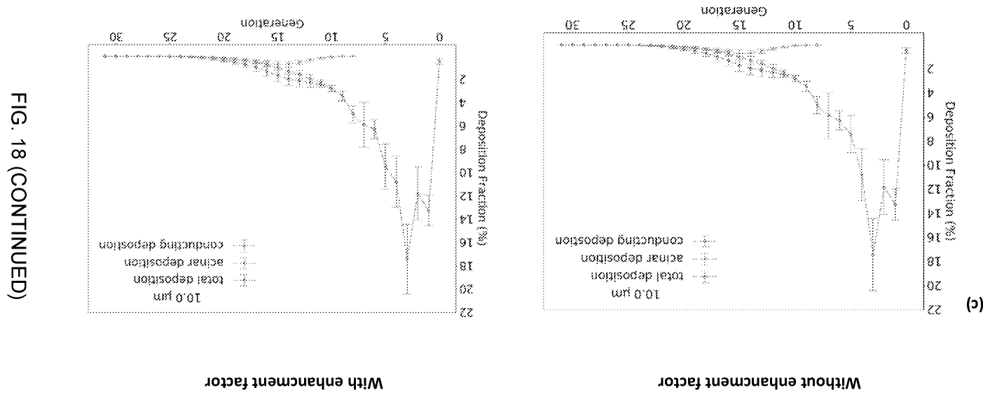

FIG. 18 further shows the distributions of deposition fraction by generation in conducting airways and acinar (respiratory) units with and without enhancement for selected particle sizes of 10.0, 1.0 and 0.01 μm. Each acinar unit is assigned a single generation number with the generation number of the terminal bronchiole attached to that unit. The features of deposition distributions for particle size from 10.0 to 0.01 μm change from large conducting deposition dominance (unimodal) to large conducting and acinar deposition dominance (bimodal), and then to small conducting and acinar deposition dominance (bimodal). Most large 10.0-μm particles are deposited in proximal large conducting airways, whereas small 0.01-μm particles are deposited in distal small airways and more are deposited in conducting region than acinar region.

### F. Validation Against CT/SPECT Data

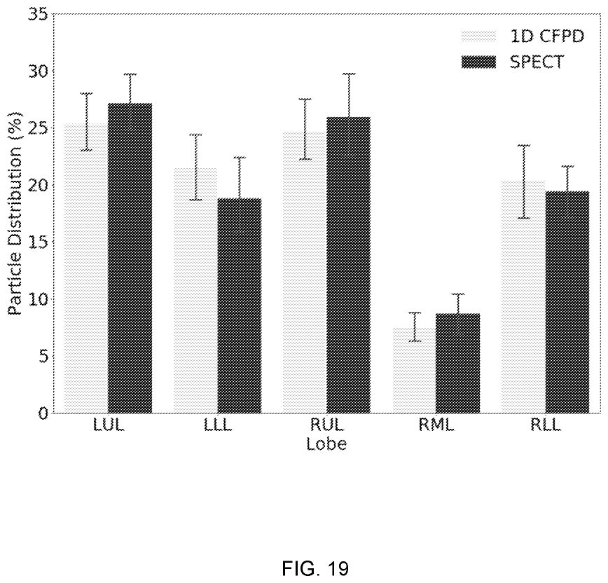

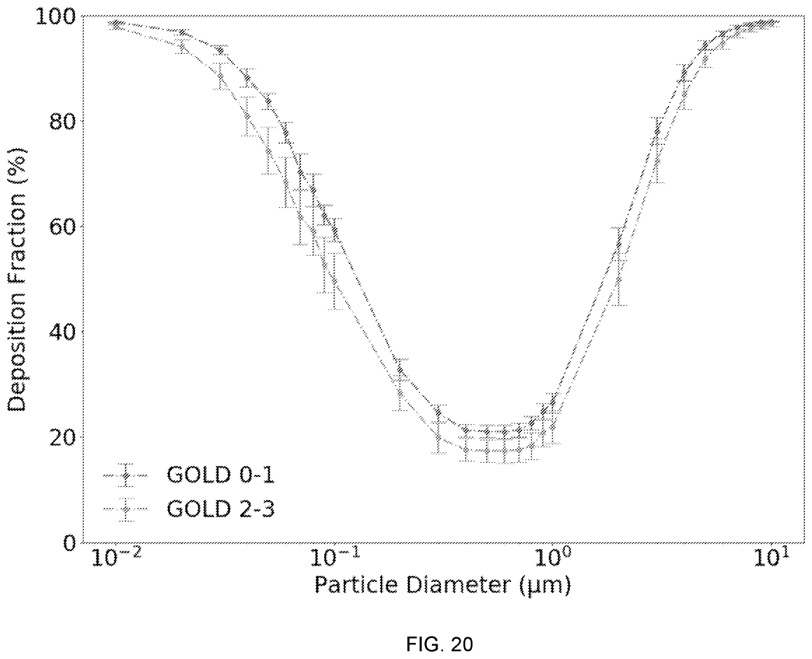

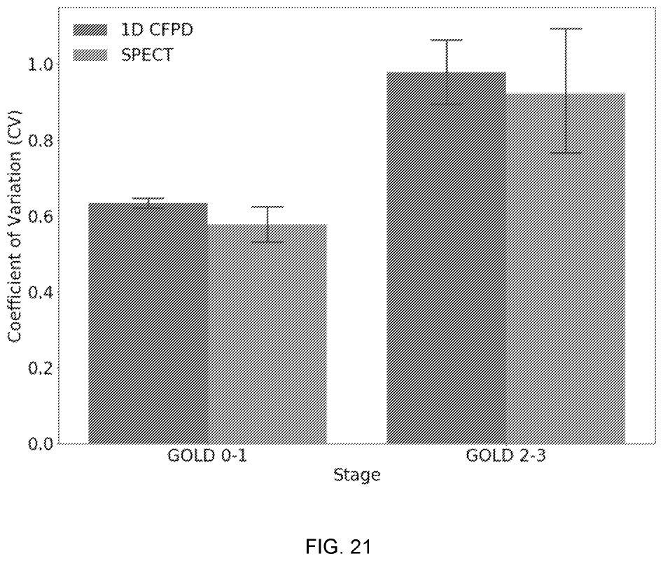

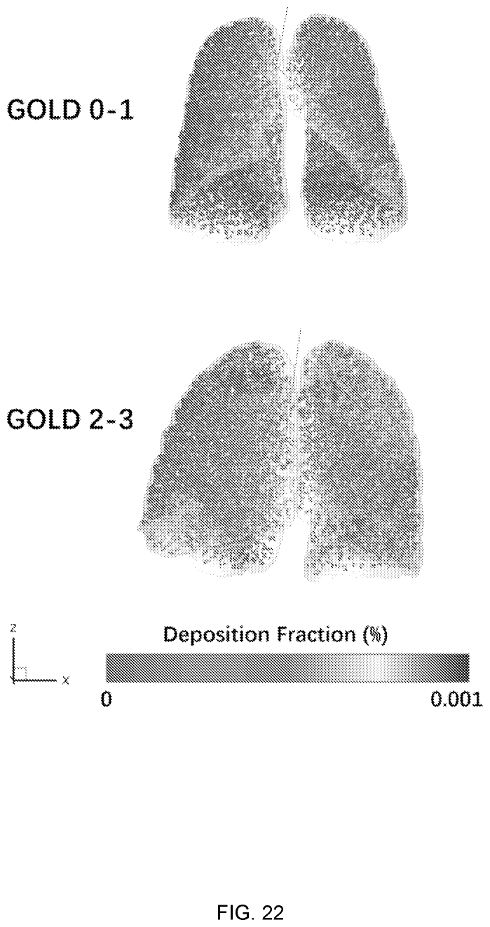

Because the size of 99mTc sulfur colloid used in SPECT imaging is below 1 μm (37), the particle diameter for the 1D CFPD simulations of the CT/SPECT subjects was set to 0.5 μm. The breathing period was set to T=4.8 s. FIG. 19 shows that the lobar deposition distributions predicted by the 1D CFPD model are highly correlated with those of the SPECT data (p>0.05). FIG. 20 further shows the deposition features of CT/SPECT subjects in subgroups for particle size ranging from 0.01 to 10.0 μm. The subjects were grouped into nonsevere-mild (GOLD 0-1) subjects and moderate-severe (GOLD 2-3) subjects. The results show that nonsevere-mild subjects had higher deposition fraction than moderate-severe COPD subjects (p<0.05), suggesting that the former may be more susceptible to environmental (pollutant) and behavioral (smoking) risks. FIG. 21 shows the coefficient of variation (CV) calculated using respective SPECT and 1D CFPD data for the two subgroups. The CV values between SPECT and 1D CFPD model (r>0.8) were in good agreement that higher CV and more heterogeneity were found in moderate-severe subjects. FIG. 22 shows the spatial deposition distributions in selected subgroup subjects, illustrating the nature of heterogeneous deposition with increasing disease severity. The ability to capture deposition heterogeneity is an important feature of a 1D subject-specific CFPD model.

### G. Discussion

Humans may breathe in particles of various size at any time. For example, grain of pollen is about 15 μm in diameter, and dust particulate matters may vary from 2.5 to 10.0 μm in diameter as denoted by PM_{2.5} and PM₁₀. Respiratory particle-laden droplets that may carry small particles, such as coronavirus generated by cough, are around 1.0-10.0 μm (45). Pharmaceutical aerosols are 5.0 μm or less (46). Bacterium is about 1.0-3.0 μm and coronavirus is about 0.1-0.5 μm. Wildfire smoke is about 0.4-0.7 μm, and electronic cigarette particles vary between 0.1-0.9 μm (47). While the bulk knowledge of particle deposition in human lungs has been established (48), the ability to quantify deposition in a subject-specific manner is desirable for the benefit-risk analysis of inhaled particles. In vivo and in vitro studies can provide real but limited data. In silico 3D CFPD is a popular method for predicting particle deposition in human lungs, however it is only limited to site studies, e.g. in an airway model of a few generations, due to high computational cost. The proposed 1D CFPD technique offers a viable option for characterizing whole-lung depositions in individuals and subpopulations with distinct lung structural and functional features.

Constructing airway structure is a key step in modeling 1D whole-lung particle deposition. Symmetric model, typical-path model and stochastic model are commonly used in 1D models. In the current 1D model, we employed a volume filling technique (30, 31) to construct CT imaging based subject-specific conducting airways and used image-registration to estimate airflow distributions to acinar units, providing initial airflow fraction input to the 1D CFD model that calculates flow fraction to each acinar unit. With airway geometry and airflow fraction generated, the deposition fraction in the whole lung was calculated using deposition probability formulae along with an enhancement factor. Comparing with dymmetric models, our model predicted higher deposition for two reasons. First, subject-specific airway structure and volume are different from hose of a symmetric model. Second, additional deposition due to the effects of secondary flow and airway geometry is accounted for by introducing an enhancement factor. When particle diameter is relatively small (<0.1 μm), particles are highly diffusive and can be easily deposited in airways. On the other side, when particles are relatively large (>1.0 μm), deposition fraction is also high because of the effect of sedimentation and impaction. For relatively small or large particle diameters, both symmetric airway model and subject-specific model show high deposition fraction. Nonetheless, the deposition fraction predicted by the subject-specific model is around 10% higher than that of symmetric model for 0.01-μm and 10.0-μm particles due to asymmetric branching and ventilation heterogeneity in the subject-specific model.

The acinar model used in the current 1D CFPD model is based on Weibel's typical-path acinar model with intra-acinar isotropic alveolar wall expansion/contraction (34). The deformation of an acinar unit and airflow fraction to each acinar unit are calculated from image registration and 1D CFD simulation. Comparing with the 3D CFPD acinar simulations of Hofemeier et al. (21), our acinar model captured the characteristics of deposition in respiratory region for tidal and deep breathing (FIG. 11). Our model also captured the features of deep inhalation in the 3D CFPD deep lung model simulations of Koullapis et al. (26) that the fraction of aerosols retained in conducting region decreases and the major deposition takes place in acinar units, resulting in higher total deposition fraction than that of tidal breathing (FIG. 12). A comparison of the deep lung model in FIG. 12 and the subject-specific whole-lung model in FIG. 17 shows that the deposition in the conducting region is dominant only in the latter because the former only simulated a single distal branch with associated higher-generation airways while the latter simulated the entire airways. FIG. 18 further shows that with decreasing particle size to 0.01 μm, the deposition in conducting region remains significant, residing in the distal small airways.

Comparing with CT/SPECT data, our 1D CFPD model captured deposition heterogeneity by lobe (FIG. 19) and by subgroups (FIG. 21). The discrepancy in the CV of deposition fraction between subgroups is correlated with the degree of air trapping, which is a disease phenotype in COPD (9). The CVs of air-trapped voxels (categorial variable) are: GOLD 0-1, 0.203±0.054 and GOLD 2-3, 0.823±0.343. Air trapping and small airway diseases in COPD hinder aerosols from reaching peripheral area. As a result, the deposition in severe subjects decreases (see FIG. 20).

There are several potential limitations in the 1D CFPD model described above. In particular, this model does not specifically consider dynamic deformation of conducting airways nor acinar morphology in diseased lungs. However, it is to be understood that the model may be modified or adjusted to consider dynamic deformation of conducting airways, acinar morphology in diseased lungs, or other such aspects.

### H. Review, Options, Variations, and Alternatives

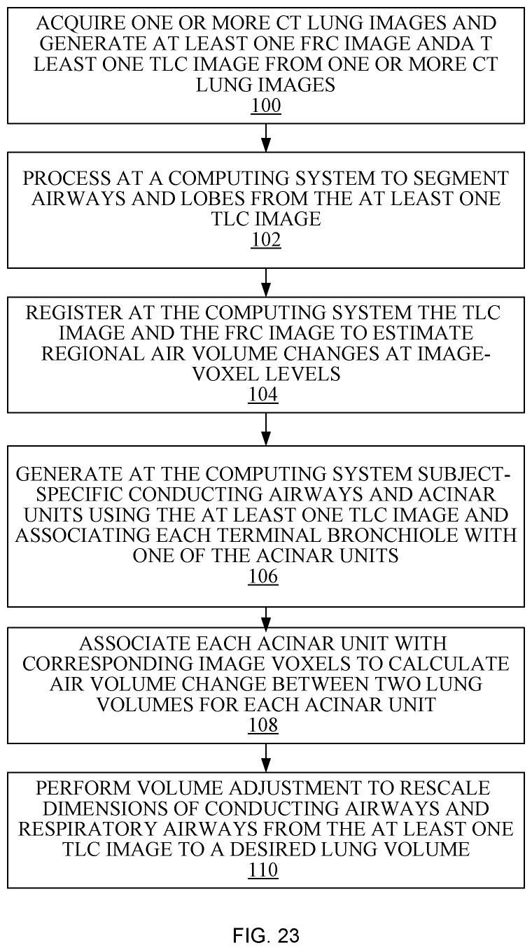

FIG. 23 illustrates a method associated with an innovative imaging-based subject-specific whole-lung deposition model. In step 100, one or more CT lung images are acquired and the method generates at least one residual functional capacity (FRC) image and at least one total lung capacity (TLC) image from the one or more CT lung images of a subject. In step 102, processing is performed at a computing system to segment airways and lobes from the at least one TLC image. At step 104, registering is performed at the computing system the at least one TLC image and the at least one FRC image to estimate regional air volume changes at image-voxel levels. At step 106, generating is performed at the computing system subject-specific conducting airways and acinar units using the at least one TLC image and associating each terminal bronchiole with one of the acinar units. At step 108, associating each acinar unit with corresponding image voxels is performed to calculate air volume change between two lung volumes for each acinar unit. At step 110, volume adjustment is performed at the computing system to rescale dimensions of conducting airways and respiratory airways from the at least one TLC image to a desired lung volume. The method may further include performing air flow modeling at the computing system using the dimensions of the conducting airways and the respiratory airways rescaled to the desired lung volume. The air flow modeling may be performed using a 1D computational fluid dynamics simulation. The method may further include performing 1D particle deposition modeling. The one or more CT lung images may include a first CT lung image acquired at inspiration and a second CT lung image acquired at expiration. The computing system may include one or more processors. The step of acquiring the one or more CT lung images may include acquiring one more CT lung images from CT scans of the subject. The method may further include generating a visual output showing results of the whole-lung modeling including the conducting airways and the respiratory airways at the desired lung volume. The whole-lung modeling may be performed for more than one dimension.

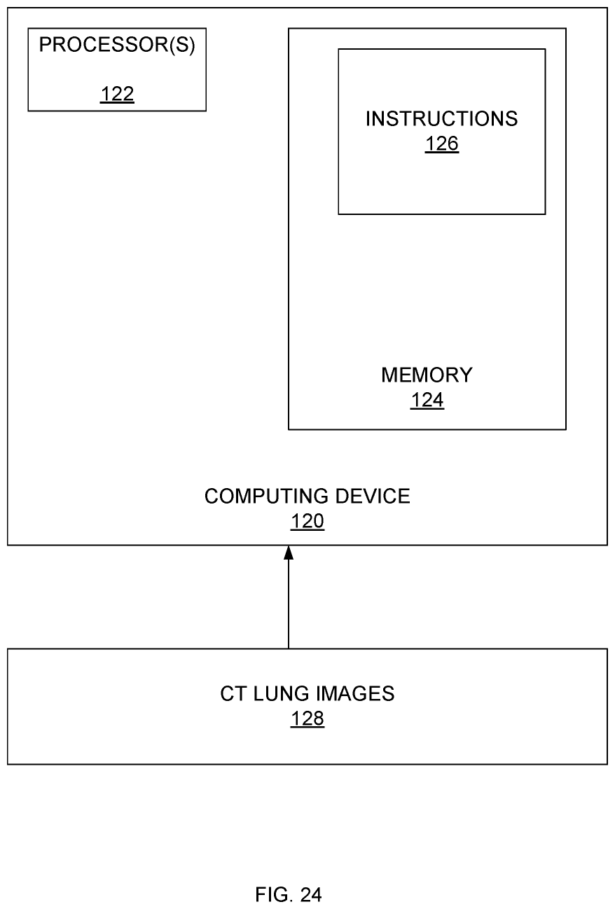

At FIG. 24, a system is provided. The system includes a computing device 120 which includes at least one processor 122, a plurality of instructions 126 for execution by the computing device 120 wherein the instructions are configured to process CT lung images 128 including at least one TLC image and at least on FRC image to segment airways and lobes from at least one TLC image, register the at least one TLC image and the at least one FRC image to estimate regional air volume changes at image-voxel levels, generate subject-specific conducting airways and acinar units using the at least one TLC image and associate each terminal bronchiole with one of the acinar units, associate each acinar unit with corresponding image voxels to calculate air volume change between two lung volumes for each acinar unit, and perform volume adjustment to rescale dimensions of conducting airways and respiratory airways from the at least one TLC image to a desired lung volume. The plurality of instructions 126 may be stored on a non-transitory machine readable medium such as memory 124. The plurality of instructions 126 may be further configured to perform air flow modeling using the dimensions of the conducting airways and the respiratory airways rescaled to the desired lung volume. The air flow modeling may be performed using a 1D computational fluid dynamics simulation. The plurality of instructions 126 may be further configured to perform particle deposition modeling. The particle deposition modeling may be one-dimensional particle deposition modeling. The one or more CT lung images 128 may include a first CT lung image acquired at inspiration and a second CT lung image acquired at expiration. The plurality of instructions 126 may further provide for generating a visual output showing results of the whole-lung modeling including the conducting airways and the respiratory airways at the desired lung volume.

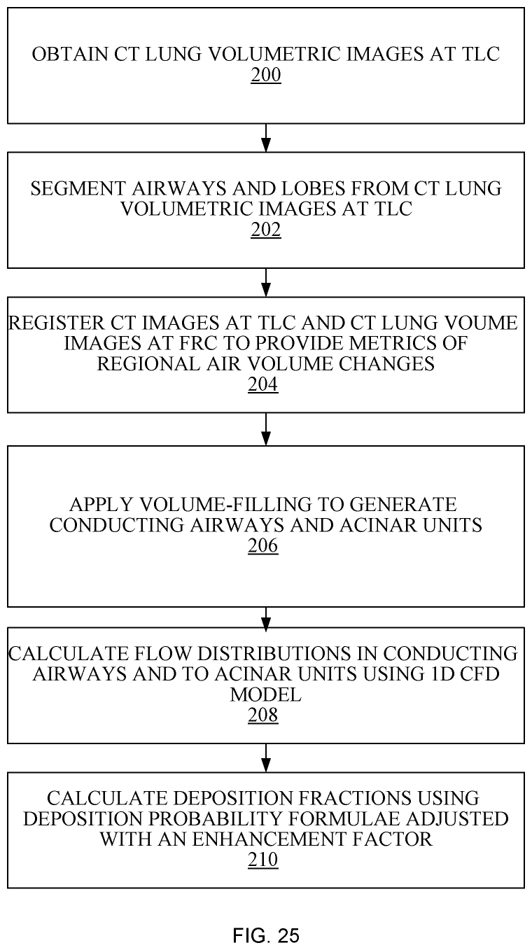

At FIG. 25, a method for generating an imaging-based subject-specific whole-lung deposition model is shown. The method includes step 200 for obtaining computed tomography (CT) lung volumetric images at total lung capacity (TLC). Step 202 provides for segmenting airways and lobes of the CT lung volumetric images at TLC. Step 204 provides for registering CT images at TLC and CT lung volume images at functional residual capacity (FRC) to provide metrics of regional air volume changes. Step 206 provides for applying volume-filling to generate conducting airways and acinar units. Step 208 provides for calculating flow distributions in conducting airways and to acinar units using a one-dimensional (1D) computational fluid dynamics (CFD) model. Step 210 provides for calculating deposition fractions using deposition probability formulae adjusted with an enhancement factor to account for effects of transient secondary flow and airway geometry to thereby provide the imaging-based subject-specific whole-lung deposition model. The steps may be performed by a computing system executing a plurality of instructions.

Although various methods, apparatus, and systems have been described throughout, it is to be understood that the present invention contemplates numerous options, variations, and alternatives as may be appropriate for use with a particular imaging technology, a particular subject, a particular application, or other factors.

For example, although emphasis has generally been on ID models, it is to be understood that multi-dimensional models also may be generated using methods and systems described herein. It is to be understood that where multi-dimensional models are used, computational complexity will increase and thus the time to produce useful resources will also increase significantly, thus in many applications, 1D models may be preferred.

In addition, it should be understood that the number of CT images acquired may vary and that some CT images at different lung volumes may be generated from existing CT images. However, it may generally be more expedient to acquire CT images directly at different lung volumes and at full inspiration and expiration.

It is to be further understood that the methods shown and described herein may be performed at a lab server or other computing system and that the methods may be implemented in software, hardware, or a combination thereof.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Reference throughout this specification to “an example” means that a particular feature, structure, or characteristic described in connection with the example is included in at least one embodiment. Thus, appearances of the phrases “in an example” in various places throughout this specification are not necessarily all referring to the same embodiment or example.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Certain embodiments may be described herein as implementing mathematical methodologies including logic or a number of components, modules, or mechanisms. Modules may constitute either software modules (e.g., code embodied on a machine-readable medium or in a transmission signal) or hardware modules. A hardware module is tangible unit capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC)) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. As used herein, “hardware-implemented module” refers to a hardware module. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time. Thus, for example, the computing device may be a hardware module.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules. Where the term “processor” is used, it is to be understood that it encompasses one or more processors whether located together or remote from one other.

Similarly, the methods described herein may be at least partially processor-implemented. For example, at least some of the operations of a method may be performed by one or processors or processor-implemented hardware modules. The performance of certain of the operations may be distributed among the one or more processors, not only residing within a single machine, but deployed across a number of machines. In some example embodiments, the processor or processors may be located in a single location, while in other embodiments the processors may be distributed across a number of locations.

The one or more processors may also operate to support performance of the relevant operations in a “cloud computing” environment or as a “software as a service” (SaaS). For example, at least some of the operations may be performed by a group of computers (as examples of machines including processors), these operations being accessible via a network (e.g., the Internet) and via one or more appropriate interfaces (e.g., application program interfaces (APIs).)

The performance of certain of the operations may be distributed among the one or more processors, not only residing within a single machine, but deployed across a number of machines. In some example embodiments, the one or more processors or processor-implemented modules may be located in a single geographic location. In other example embodiments, the one or more processors or processor-implemented modules may be distributed across a number of geographic locations.

Some portions of this specification are presented in terms of algorithms or symbolic representations of operations on data stored as bits or binary digital signals within a machine memory (e.g., a computer memory). These algorithms or symbolic representations are examples of techniques used by those of ordinary skill in the data processing arts to convey the substance of their work to others skilled in the art. As used herein, an “algorithm” is a self-consistent sequence of operations or similar processing leading to a desired result. In this context, algorithms and operations involve physical manipulation of physical quantities. Typically, but not necessarily, such quantities may take the form of electrical, magnetic, or optical signals capable of being stored, accessed, transferred, combined, compared, or otherwise manipulated by a machine. It is convenient at times, principally for reasons of common usage, to refer to such signals using words such as “data,” “content,” “bits,” “values,” “elements,” “symbols,” “characters,” “terms,” “numbers,” “numerals,” or the like. These words, however, are merely convenient labels and are to be associated with appropriate physical quantities.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the disclosure. This description should be read to include one or at least one and the singular also includes the plural unless it is obvious that it is meant otherwise.

The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Similarly, if a method is described herein as comprising a series of steps, the order of such steps as presented herein is not necessarily the only order in which such steps may be performed, and certain of the stated steps may possibly be omitted and/or certain other steps not described herein may possibly be added to the method.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Reference throughout this specification to “an example” means that a particular feature, structure, or characteristic described in connection with the example is included in at least one embodiment. Thus, appearances of the phrases “in an example” in various places throughout this specification are not necessarily all referring to the same embodiment or example.

The invention is not to be limited to the particular embodiments described herein. In particular, the invention contemplates numerous variations in the specific methodology used and structures provided as described herein. The foregoing description has been presented for purposes of illustration and description. It is not intended to be an exhaustive list or limit any of the invention to the precise forms disclosed. It is contemplated that other alternatives or exemplary aspects are considered included in the invention. The description is merely examples of embodiments, processes, or methods of the invention. It is understood that any other modifications, substitutions, and/or additions can be made, which are within the intended spirit and scope of the invention.

### REFERENCES

The following references are hereby incorporated by reference in their entireties.
1. Smith B M, Traboulsi H, Austin J H M, Manichaikul A, Hoffman E A, Bleecker E R, Cardoso W V, Cooper C, Couper D J, Dashnaw S M, Guo J, Han M K, Hansel N N, Hughes E W, Jacobs D R, Kanner R E, Kaufman J D, Kleerup E, Lin C-L, Liu K, Lo Cascio C M, Martinez F J, Nguyen J N, Prince M R, Rennard S, Rich S S, Simon L, Sun Y, Watson K E, Woodruff P G, Baglole C J, Barr R G, Lung ftM, investigators S. Human airway branch variation and chronic obstructive pulmonary disease. Proceedings of the National Academy of Sciences. 2018; 115(5):E974-E81. doi: 10.1073/pnas.1715564115.
2. Smith B M, Kirby M, Hoffman E A, Kronmal R A, Aaron S D, Allen N B, Bertoni A, Coxson H O, Cooper C, Couper D J, Criner G, Dransfield M T, Han M K, Hansel N N, Jacobs D R, Jr., Kaufman J D, Lin C L, Manichaikul A, Martinez F J, Michos E D, Oelsner E C, Paine R, 3rd, Watson K E, Benedetti A, Tan W C, Bourbeau J, Woodruff P G, Barr R G. Association of Dysanapsis With Chronic Obstructive Pulmonary Disease Among Older Adults. Jama. 2020; 323(22):2268-80. Epub 2020/06/10. doi: 10.1001/jama.2020.6918. PubMed PMID: 32515814; PMCID: PMC7284296
3. Tanabe N, Sato S, Oguma T, Shima H, Sato A, Muro S, Hirai T. Associations of airway tree to lung volume ratio on computed tomography with lung function and symptoms in chronic obstructive pulmonary disease. Respiratory Research. 2019; 20(1):77. doi: 10.1186/si2931-019-1047-5.
4. Lin C-L, Choi S, Haghighi B, Choi J, Hoffman E A. Cluster-Guided Multiscale Lung Modeling via Machine Learning. In: Andreoni W, Yip S, editors. Handbook of Materials Modeling: Applications: Current and Emerging Materials. Cham: Springer International Publishing; 2018. p. 1-20.
5. Castaldi P J, Boueiz A, Yun J, Estepar R S J, Ross J C, Washko G, Cho M H, Hersh C P, Kinney G L, Young K A, Regan E A, Lynch D A, Criner G J, Dy J G, Rennard S I, Casaburi R, Make B J, Crapo J, Silverman E K, Hokanson J E. Machine Learning Characterization of COPD Subtypes: Insights From the COPDGene Study. Chest. 2020; 157(5):1147-57. Epub 2019/12/31. doi: 10.1016/j.chest.2019.11.039. PubMed PMID: 31887283; PMCID: PMC7242638.
6. Lin C-L, Hoffman E A, Kassinos S. Chapter 14—Machine learning and in silico methods. In: Kassinos S, Backman P, Conway J, Hickey A J, editors. Inhaled Medicines: Academic Press; 2021. p. 375-90.
7. Haghighi B, Choi S, Choi J, Hoffman E A, Comellas A P, Newell J D, Graham Barr R, Bleecker E, Cooper C B, Couper D, Han M L, Hansel N N, Kanner R E, Kazerooni E A, Kleerup E A C, Martinez F J, O'Neal W, Rennard S I, Woodruff P G, Lin C-L. Imaging-based clusters in current smokers of the COPD cohort associate with clinical characteristics: the SubPopulations and Intermediate Outcome Measures in COPD Study (SPIROMICS). Respiratory Research. 2018; 19(1):178. doi: 10.1186/s12931-018-0888-7.
8. Haghighi B, Choi S, Choi J, Hoffman E A, Comellas A P, Newell J D, Lee C H, Barr R G, Bleecker E, Cooper C B, Couper D, Han M L, Hansel N N, Kanner R E, Kazerooni E A, Kleerup E A C, Martinez F J, O'Neal W, Paine R, Rennard S I, Smith B M, Woodruff P G, Lin C-L. Imaging-based clusters in former smokers of the COPD cohort associate with clinical characteristics: the SubPopulations and intermediate outcome measures in COPD study (SPIROMICS). Respiratory Research. 2019; 20(1):153. doi: 10.1186/s12931-019-1121-z.
9. Zou C, Li F, Choi J, Haghighi B, Choi S, Rajaraman P K, Comellas A P, Newell J D, Lee C H, Barr R G, Bleecker E, Cooper C B, Couper D, Han M, Hansel N N, Kanner R E, Kazerooni E A, Kleerup E C, Martinez F J, O'Neal W, Paine R, 3rd, Rennard S I, Smith B M, Woodruff P G, Hoffman E A, Lin C L. Longitudinal Imaging-Based Clusters in Former Smokers of the COPD Cohort Associate with Clinical Characteristics: The SubPopulations and Intermediate Outcome Measures in COPD Study (SPIROMICS). Int J Chron Obstruct Pulmon Dis. 2021; 16:1477-96. Epub 2021/06/10. doi: 10.2147/copd.S301466. PubMed PMID: 34103907; PMCID: PMC8178702.
10. Li F, Choi J, Zou C, Newell J D, Comellas A P, Lee C H, Ko H, Barr R G, Bleecker E R, Cooper C B, Abtin F, Barjaktarevic I, Couper D, Han M, Hansel N N, Kanner R E, Paine R, Kazerooni E A, Martinez F J, O'Neal W, Rennard S I, Smith B M, Woodruff P G, Hoffman E A, Lin C-L. Latent traits of lung tissue patterns in former smokers derived by dual channel deep learning in computed tomography images. Scientific Reports. 2021; 11(1):4916. doi: 10.1038/s41598-021-84547-5.
11. Choi S, Hoffman E A, Wenzel S E, Castro M, Fain S, Jarjour N, Schiebler M L, Chen K, Lin C-L. Quantitative computed tomographic imaging-based clustering differentiates asthmatic subgroups with distinctive clinical phenotypes. Journal of Allergy and Clinical Immunology. 2017; 140(3):690-700.e8. doi: https://doi.org/10.1016/j.jaci.2016.11.053.
12. Choi J, LeBlanc L J, Choi S, Haghighi B, Hoffman E A, O'Shaughnessy P, Wenzel S E, Castro M, Fain S, Jarjour N, Schiebler M L, Denlinger L, Delvadia R, Walenga R, Babiskin A, Lin C-L. Differences in Particle Deposition Between Members of Imaging-Based Asthma Clusters. Journal of Aerosol Medicine and Pulmonary Drug Delivery. 2019; 32(4):213-23. doi: 10.1089/jamp.2018.1487.
13. Rajaraman P K, Choi J, Hoffman E A, O'Shaughnessy P T, Choi S, Delvadia R, Babiskin A, Walenga R, Lin C-L. Transport and deposition of hygroscopic particles in asthmatic subjects with and without airway narrowing. Journal of Aerosol Science. 2020; 146:105581. doi: https://doi.org/10.1016/j.jaerosci.2020.105581.
14. Hofmann W. Modelling inhaled particle deposition in the human lung—A review. Journal of Aerosol Science. 2011; 42(10):693-724. doi: https://doi.org/10.1016/j.jaerosci.2011.05.007.
15. Yeh H-C, Schum G M. Models of human lung airways and their application to inhaled particle deposition. Bulletin of Mathematical Biology. 1980; 42(3):461-80. doi: https://doi.org/10.1016/S0092-8240(80)80060-7.
16. Raabe O G, Yeh, H C, Schum, G M and Phalen, R F. Tracheobronchial Geometry Human, Dog, Rat. Albuquerque, New Mexico, USA: 1976.
17. Hofmann W, Koblinger L. Monte Carlo modeling of aerosol deposition in human lungs. Part II: Deposition fractions and their sensitivity to parameter variations. Journal of Aerosol Science. 1990; 21(5):675-88. doi: https://doi.org/10.1016/0021-8502(90)90122-E.
18. Koblinger L, Hofmann W. Monte Carlo modeling of aerosol deposition in human lungs. Part I: Simulation of particle transport in a stochastic lung structure. Journal of Aerosol Science. 1990; 21(5):661-74. doi: https://doi.org/10.1016/0021-8502(90)90121-D.
19. Schum M, Yeh H-C. Theoretical evaluation of aerosol deposition in anatomical models of mammalian lung airways. Bulletin of mathematical biology. 1980; 42(1):1-15.
20. Asgharian B, Hofmann W, Bergmann R. Particle Deposition in a Multiple-Path Model of the Human Lung. Aerosol Science and Technology. 2001; 34(4):332-9. doi: 10.1080/02786820119122.
21. Hofemeier P, Koshiyama K, Wada S, Sznitman J. One (sub-)acinus for all: Fate of inhaled aerosols in heterogeneous pulmonary acinar structures. Eur J Pharm Sci. 2018; 113:53-63. Epub 2017/09/28. doi: 10.1016/j.ejps.2017.09.033. PubMed PMID: 28954217; PMCID: PMC5810902.
22. Hofemeier P, Sznitman J. Revisiting pulmonary acinar particle transport: convection, sedimentation, diffusion, and their interplay. Journal of Applied Physiology. 2015; 118(11):1375-85. doi: 10.1152/japplphysiol.01117.2014.
23. Koshiyama K, Wada S. Mathematical model of a heterogeneous pulmonary acinus structure. Computers in Biology and Medicine. 2015; 62:25-32. doi: https://doi.org/10.1016/j.compbiomed.2015.03.032.
24. Ochs M, Nyengaard J R, Jung A, Knudsen L, Voigt M, Wahlers T, Richter J, Gundersen H J. The number of alveoli in the human lung. Am J Respir Crit Care Med. 2004; 169(1):120-4. Epub 2003/09/27. doi: 10.1164/rccm.200308-11070C. PubMed PMID: 14512270.
25. Koullapis P G, Hofemeier P, Sznitman J, Kassinos S C. An efficient computational fluid-particle dynamics method to predict deposition in a simplified approximation of the deep lung. Eur J Pharm Sci. 2018; 113:132-44. Epub 2017/09/18. doi: 10.1016/j.ejps.2017.09.016. PubMed PMID: 28917963.
26. Koullapis P G, Stylianou F S, Sznitman J, Olsson B, Kassinos S C. Towards whole-lung simulations of aerosol deposition: A model of the deep lung. Journal of Aerosol Science. 2020; 144:105541. doi: https://doi.org/10.1016/j.jaerosci.2020.105541.
27. Lippmann M. Regional Deposition of Particles in the Human Respiratory Tract. Comprehensive Physiology. p. 213-32.
28. Stahlhofen W, Rudolf G, James A C. Intercomparison of Experimental Regional Aerosol Deposition Data. Journal of Aerosol Medicine. 1989; 2(3):285-308. doi: 10.1089/jam.1989.2.285.
29. De Backer J W, Vos W G, Vinchurkar S C, Claes R, Drollmann A, Wulfrank D, Parizel P M, Germonpre P, De Backer W. Validation of computational fluid dynamics in CT-based airway models with SPECT/CT. Radiology. 2010; 257(3):854-62. Epub 2010/11/19. doi: 10.1148/radiol.10100322. PubMed PMID: 21084417.
30. Tawhai M H, Hunter P, Tschirren J, Reinhardt J, McLennan G, Hoffman E A. CT-based geometry analysis and finite element models of the human and ovine bronchial tree. J Appl Physiol (1985). 2004; 97(6):2310-21. Epub 2004 Aug. 24. doi: 10.1152/japplphysiol.00520.2004. PubMed PMID: 15322064.
31. Tawhai M H, Pullan A J, Hunter P J. Generation of an Anatomically Based Three-Dimensional Model of the Conducting Airways. Annals of Biomedical Engineering. 2000; 28(7):793-802. doi: 10.1114/1.1289457.
32. Youbin Yin E A H, Ching-Long Lin. Mass preserving nonrigid registration of CT lung images using cubic B-spline. Medical physics. 2009; 36,9 (2009): 4213-22. doi: 10.1118/1.3193526.
33. Yin Y, Choi J, Hoffman E A, Tawhai M H, Lin C L. Simulation of pulmonary air flow with a subject-specific boundary condition. J Biomech. 2010; 43(11):2159-63. Epub 2010/05/21. doi: 10.1016/j.jbiomech.2010.03.048. PubMed PMID: 20483412; PMCID: PMC3034656.
34. Weibel E R, Sapoval B, Filoche M. Design of peripheral airways for efficient gas exchange. Respir Physiol Neurobiol. 2005; 148(1-2):3-21. Epub 2005/06/01. doi: 10.1016/j.resp.2005.03.005. PubMed PMID: 15921964.
35. Choi S, Yoon S, Jeon J, Zou C, Choi J, Tawhai M H, Hoffman E A, Delvadia R, Babiskin A, Walenga R, Lin C-L. ID network simulations for evaluating regional flow and pressure distributions in healthy and asthmatic human lungs. Journal of Applied Physiology. 2019; 127(1):122-33. doi: 10.1152/japplphysiol.00016.2019. PubMed PMID: 31095459.
36. Yoon S, Tam T M, Rajaraman P K, Lin C-L, Tawhai M, Hoffman E A, Choi S. An integrated 1D breathing lung simulation with relative hysteresis of airway structure and regional pressure for healthy and asthmatic human lungs. Journal of Applied Physiology. 2020; 129(4):732-47. doi: 10.1152/japplphysiol.00176.2020. PubMed PMID: 32758040.
37. Krogsgaard O. Technetium-99m-sulfur colloid. European Journal of Nuclear Medicine. 2004; 1:31-5.
38. Sirr S A, Juenemann P J, Tom H, Boudreau R J, Chandler R P, Loken M K. Effect of ethanol on droplet size, efficiency of delivery, and clearance characteristics of technetium-99m DTPA aerosol. J Nucl Med. 1985; 26(6):643-6. Epub 1985/06/01. PubMed PMID: 3889235.
39. Lin C-L, Tawhai M H, McLennan G, Hoffman E A. Characteristics of the turbulent laryngeal jet and its effect on airflow in the human intra-thoracic airways. Respiratory Physiology & Neurobiology. 2007; 157(2):295-309. doi: https://doi.org/10.1016/j.resp.2007.02.006.
40. Miyawaki S, Hoffman E A, Lin C-L. Numerical simulations of aerosol delivery to the human lung with an idealized laryngeal model, image-based airway model, and automatic meshing algorithm. Computers & Fluids. 2017; 148:1-9. doi: https://doi.org/10.1016/j.compfluid.2017.02.008.
41. Hofmann W, Martonen T B. The Significance of Enhanced Radionuclide Deposition at Bronchial Bifurcations. The Annals of Occupational Hygiene. 1988; 32(inhaled_particles_VI):1055-65. doi: 10.1093/annhyg/32.inhaled_particles_VI.1055.
42. Yin Y, Hoffman E A, Lin C-L. Mass preserving nonrigid registration of CT lung images using cubic B-spline. Medical physics. 2009; 36,9 (2009): 4213-22. doi: 10.1118/1.3193526.
43. Haghighi B, D. Ellingwood N, Yin Y, Hoffman E A, Lin C-L. A GPU-based symmetric non-rigid image registration method in human lung. Medical & Biological Engineering & Computing. 2018; 56(3):355-71. doi: 10.1007/s11517-017-1690-2.
44. Fuller B M F I, Mohr N M, et al. Lung-Protective Ventilation Initiated in the Emergency Department (LOV-ED): A Quasi-Experimental, Before-After Trial. Ann Emerg Med. 2017. doi: doi:10.1016/j.annemergmed.2017.01.013.
45. Madas B G, Füri P, Farkas Á, Nagy A, Czitrovszky A, Bálisházy I, Schay G G, Horváth A. Deposition distribution of the new coronavirus (SARS-CoV-2) in the human airways upon exposure to cough-generated droplets and aerosol particles. Scientific Reports. 2020; 10(1):22430. doi: 10.1038/s41598-020-79985-6.
46. Newman S P. AEROSOLS. In: Laurent G J, Shapiro S D, editors. Encyclopedia of Respiratory Medicine. Oxford: Academic Press; 2006. p. 58-64.
47. Mulder H A, Patterson J L, Halquist M S, Kosmider L, Turner J B M, Poklis J L, Poklis A, Peace M R. The Effect of Electronic Cigarette User Modifications and E-liquid Adulteration on the Particle Size Profile of an Aerosolized Product. Scientific Reports. 2019; 9(1):10221. doi: 10.1038/s41598-019-46387-2.
48. Darquenne C. Deposition Mechanisms. Journal of Aerosol Medicine and Pulmonary Drug Delivery. 2020; 33(4):181-5. doi: 10.1089/jamp.2020.29029.cd. PubMed PMID: 32598200.

What is claimed is:

1. A method for providing a CT imaging-based subject-specific whole-lung modelling, the method comprising steps of:
acquiring one or more CT lung images and generating at least one residual functional capacity (FRC) image and at least one total lung capacity (TLC) image from the one or more CT lung images of a subject;
processing at a computing system to segment airways and lobes from the at least one TLC image;
registering at the computing system the at least one TLC image and the at least one FRC image to estimate regional air volume changes at image-voxel levels;
generating at the computing system subject-specific conducting airways and acinar units using the at least one TLC image and associating each terminal bronchiole with one of the acinar units;
associating each acinar unit with corresponding image voxels to calculate air volume change between two lung volumes for each acinar unit;
performing at the computing system volume adjustment to rescale dimensions of conducting airways and respiratory airways from the at least one TLC image to a desired lung volume.

2. The method of claim 1 further comprising performing air flow modeling at the computing system using the dimensions of the conducting airways and the respiratory airways rescaled to the desired lung volume.

3. The method of claim 2 wherein the air flow modeling is performed using a 1D computational fluid dynamics simulation.

4. The method of claim 2 further comprising performing at the computing system 1D particle deposition modeling.

5. The method of claim 1 wherein the one or more CT lung images including a first CT lung image acquired at inspiration and a second CT lung image acquired at expiration.

6. The method of claim 1 wherein the computing system comprises one or more processors.

7. The method of claim 1 wherein the acquiring the one or more CT lung images comprises acquiring one more CT lung images from CT scans of the subject.

8. The method of claim 1 further comprising generating a visual output showing results of the whole-lung modeling including the conducting airways and the respiratory airways at the desired lung volume.

9. The method of claim 1 wherein the whole-lung modeling is performed for more than one dimension.

10. A system comprising: a computing device comprising at least one processor; a plurality of instructions for execution by the computing device wherein the instructions are configured to process CT lung images including at least one TLC image and at least one FRC image to segment airways and lobes from at least one TLC image, register the at least one TLC image and the at least one FRC image to estimate regional air volume changes at image-voxel levels, generate subject-specific conducting airways and acinar units using the at least one TLC image and associate each terminal bronchiole with one of the acinar units, associate each acinar unit with corresponding image voxels to calculate air volume change between two lung volumes for each acinar unit, and perform volume adjustment to rescale dimensions of conducting airways and respiratory airways from the at least one TLC image to a desired lung volume.

11. The system of claim 10 wherein the plurality of instructions are stored on a non-transitory machine readable medium.

12. The system of claim 10 wherein the plurality of instructions are further configured to perform air flow modeling using the dimensions of the conducting airways and the respiratory airways rescaled to the desired lung volume.

13. The system of claim 12 wherein the air flow modeling is performed using a 1D computational fluid dynamics simulation.

14. The system of claim 12 wherein the plurality of instructions are further configured to perform particle deposition modeling.

15. The system of claim 14 wherein the particle deposition modeling is one-dimensional particle deposition modeling.

16. The system of claim 10 wherein the one or more CT lung images include a first CT lung image acquired at inspiration and a second CT lung image acquired at expiration.

17. The system of claim 10 wherein the plurality of instructions further provide for generating a visual output showing results of whole-lung modeling including the conducting airways and the respiratory airways at the desired lung volume.

18. A method for generating an imaging-based subject-specific whole-lung deposition model comprises steps of:
obtaining computed tomography (CT) lung volumetric images at total lung capacity (TLC);
segment airways and lobes from the CT lung volumetric images at TLC;
register CT images at TLC and CT lung volume images at functional residual capacity (FRC) to provide metrics of regional air volume changes;
applying volume-filling to generate conducting airways and acinar units;
calculating flow distributions in conducting airways and to acinar units using a one-dimensional (1D) computational fluid dynamics (CFD) model;
calculating deposition fractions using deposition probability formulae adjusted with an enhancement factor to account for effects of transient secondary flow and airway geometry to thereby provide the imaging-based subject-specific whole-lung deposition model.

19. The method of claim 18 wherein the steps are performed by a computing system executing a plurality of instructions using at least one processor.

20. The method of claim 19 further comprising generating a visual output of the imaging-based subject-specific whole-lung deposition model on a screen display.
